(19) United States
(12) Patent Application Publication
(10) Pub. No.: US 2004/0259777 A1
(43) Pub. Date: Dec. 23, 2004

(54) Method of treating metabolic disorders using neuronatin polypeptides

(76) Inventors: Kenneth Thirstrup, Kobenhavn NV (DK); Jens Damsgaard Mikkelsen, Lyngby (DK); Niels Vrang, Kobenhavn (DK); Leif Kongskov Larsen, Rodovre (DK); Mads Tang-Christensen, Kobenhaven O (DK)

Correspondence Address: BIRCH STEWART KOLASCH & BIRCH, PO BOX 747, FALLS CHURCH, VA, 22040-0747, US

(21) Appl. No.: 10/479,580

(22) PCT Filed: Jun. 7, 2002
(86) PCT No.: PCT/DK02/00384

(30) Foreign Application Priority Data
Jun. 7, 2001 (DK) PA 2001 00889

(51) Int. Cl. A61K 38/22

(52) U.S. Cl. 514/12

(57) ABSTRACT

The present invention relates generally to the control of metabolic disorders, such as body weight disorders, of animals including mammals and humans, and more particularly to polypeptides identified herein as modulators of metabolic disorders, and to diagnostic and therapeutic uses of such modulators. In its broadest aspect, the present invention relates to mammalian neuronatin proteins sequences, isoforms thereof, and degenerate variations thereof, that demonstrate the ability to participate in the control of mammalian metabolism and that have been postulated to play a critical role in the regulation of body weight, adiposity and non-insulin dependent diabetes mellitus (NIDDM). The invention further relates to the preparation of the modulators of the invention.

### TECHNICAL FIELD

The present invention relates to a method of preventing or treating metabolic disorders, such as body weight and non-insulin dependent diabetes mellitus, of an animal using neuronatin polypeptides.

### BACKGROUND ART

Obesity, defined as an excess of body fat relative to lean body mass, is associated with important psychological and medical morbidities, the latter including hypertension, elevated blood lipids, and Type II or non-insulin-dependent diabetes mellitus (NIDDM). Obesity, and especially upper body obesity, is the most common nutritional disorder of the world. Numerous studies indicate that lowering body weight dramatically reduces risk for chronic diseases, such as diabetes, hypertension, hyperlipidaemia, coronary heart disease, and musculo-skeletal diseases. For example, various measures of obesity, including, simple body weight, waist-to-hip ratios, and mesenteric fat depots, are strongly correlated with risk for non-insulin dependent diabetes (NIDDM), also known as type II diabetes. According to the American Diabetes Association (1995) about 80% of NIDDM patients are overweight. Weight-reduction is a specific goal of medical treatment of many chronic diseases, including NIDDM.

Current methods for promoting weight loss are not completely satisfactory. Some obese patients may lose weight through deliberate modification of behaviour, such as changing diet and increased exercise. Failure to achieve weight loss by these methods may be due to genetic factors that cause increased appetite, a preference for high-fat foods, or a tendency for lipogenic metabolism. Failure to achieve weight loss by any measure is extremely common, and the estimated 33 billion dollars spent yearly on weight-loss measures are largely futile. Thus, new methods and compositions such as pharmaceutical agents that promote weight-loss are urgently needed to complement old approaches.

It is well known that the brain exerts overall control of energy intake and expenditure. The hypothalamus plays a central role in this by forming links between the central and autonomic nervous systems and between the nervous system and the endocrine glands. To be able to regulate body weight the brain must perform two vital functions. First it must be able to sense the size of adipose mass and energy expenditure as a mean to maintain body mass at a certain level. One way that the brain can adjust energy intake and energy expenditure is by altering neuropeptide production. Many neuropeptides influence energy balance by selectively stimulating either the sympathetic or parasympathetic nervous system.

The discovery of leptin by Friedman et al. in 1994 (see e.g. Zhang Y, Proenca R, Maffei M, Barone M, Leopold L, & Friedman J M: Positional cloning of the mouse obese gene and its human homologue; Nature 1994 372, 425-432) was a key event in recent obesity research. This confirmed the lipostatic theory in the sense that leptin was identified as a peripheral signal capable of signalling size of adipose tissue stores to the brain. It was soon discovered that leptin alone is not sufficient as a treatment against obesity, as obese people in many cases exhibit a high leptin concentration and are resistant to leptin treatment. A number of neuropeptides have been found which regulates body weight and many of these are regulated in response to leptin. Neuropeptide Y (NPY) and Agouti related peptide (AgRP) are both down-regulated by leptin and up-regulated in leptin-deficient animals, whereas cocaine- and amphetamine-regulated transcript (CART) and hypothalamic pro-opiomelanocortin (POMC) are up-regulated in response to leptin and down-regulated in leptin-deficient animals. Via these routes leptin exerts a net inhibition of food intake. All these peptides and their corresponding receptors are regarded as pharmaceutical targets in the modulation of feeding behaviour. It is therefore clear that new additional neuropeptides modulated by leptin and present in the hypothalamus may possess the same important features as the above-mentioned neuropeptides.

U.S. Pat. No. 5,837,535 discloses the polynucleotide and protein sequence of α-neuronatin and β-neuronatin from rat and human.

### SUMMARY OF THE INVENTION

The present invention relates to the following subject matter:

A method of preventing or treating a metabolic disorder of an animal comprising administering to the animal an effective amount of 1) a polypeptide according to SEQ ID NO: 3 α-neuronatin) or SEQ ID NO: 4 (β-neuronatin), 2) a variant of one of the said sequences having at least 50% identity therewith, 3) a fragment of SEQ ID NO: 3 or SEQ ID NO: 4 comprising at least 7 amino acids, or 4) a variant of the said fragment having at least 80% identity therewith.

A method of preventing or treating a metabolic disorder in an animal comprising administering to the animal an effective amount of a polypeptide encoded by 1) a polynucleotide according to SEQ ID NO: 1 (α-neuronatin) or SEQ ID NO: 2 (1-neuronatin), 2) a variant of one of the said sequences having at least 30% identity therewith, 3) a fragment of SEQ ID NO: 1 or SEQ ID NO: 2 comprising at least 21 nucleotides, 4) a variant of one of the said fragments having at least 60% identity therewith, or 5) a sequence, which hybridises with SEQ ID NO: 1 or SEQ ID NO: 2 under highly stringent conditions.

A polypeptide having the sequence of 1) the fragment of SEQ ID NO: 3 consisting of amino acids 38-81, 2) the fragment of SEQ ID NO: 4 consisting of amino acids 32-54, or 3) a variant of one of the said sequences having at least 50% identity therewith.

A polypeptide for preventing or treating a metabolic disorder in an animal, wherein the polypeptide is 1) a polypeptide according to SEQ ID NO: 3 (α-neuronatin) or SEQ ID NO: 4 (β-neuronatin), 2) a variant of one of the said sequences having at least 50% identity therewith, 3) a fragment of SEQ ID NO: 3 or SEQ ID NO: 4 comprising at least 7 amino acids, or 4) a variant of the said fragment having at least 80% identity therewith.

Use of 1) a polypeptide according to SEQ ID NO: 3 (α-neuronatin) or SEQ ID NO: 4 (β-neuronatin), 2) a variant of one of the said sequences having at least 50% identity therewith, 3) a fragment of SEQ ID NO: 3 or SEQ ID NO: 4 comprising at least 7 amino acids, or 4) a variant of the said fragment having at least 80% identity therewith, for the manufacture of a pharmaceutical composition for preventing or treating a metabolic disorder in an animal.

A pharmaceutical composition comprising as an active substance 1) a polypeptide according to SEQ ID NO: 3 (α-neuronatin) or SEQ ID NO: 4 (β-neuronatin), 2) a variant of one of the said sequences having at least 50% identity therewith, 3) a fragment of SEQ ID NO: 3 or SEQ ID NO: 4 comprising at least 7 amino acids, or 4) a variant of the said fragment having at least 80% identity therewith.

A pharmaceutical composition for preventing or treating metabolic disorders comprising as an active substance 1) a polypeptide according to SEQ ID NO: 3 α-neuronatin) or SEQ ID NO: 4 (β-neuronatin), 2) a variant of one of the said sequences having at least 50% identity therewith, 3) a fragment of SEQ ID NO: 3 or SEQ ID NO: 4 comprising at least 7 amino acids, or 4) a variant of the said fragment having at least 80% identity therewith.

A pharmaceutical composition comprising as an active substance a stimulator to a polypeptide according to SEQ ID NO: 3 (α-neuronatin) or SEQ ID NO: 4 (β-neuronatin), or a natural occurring variant thereof.

A pharmaceutical composition comprising as an active substance an inhibitor to a polypeptide according to SEQ ID NO: 3 (α-neuronatin) or SEQ ID NO: 4 (β-neuronatin), or a natural occurring variant thereof.

A pharmaceutical composition comprising as an active substance an inhibitor to a polynucleotide according to SEQ ID NO: 1 (α-neuronatin) or SEQ ID NO: 2 (β-neuronatin), or a natural occurring variant thereof.

A method of diagnosing or prognosticating a metabolic disorder in an animal comprising determining the sequence of the polynucleotide, which encodes neuronatin, by the use of 1) a polynucleotide according to SEQ ID NO: 1 α-neuronatin) or SEQ ID NO: 2 (β-neuronatin), 2) a variant of one of the said sequences having at least 30% identity therewith, 3) a fragment of SEQ ID NO: 1 or SEQ ID NO: 2 comprising at least 21 nucleotides, 4) a variant of one of the said fragments having at least 60% identity therewith, or 5) a sequence, which hybridises with SEQ ID NO: 1 or SEQ ID NO: 2 under highly stringent conditions, and comparing the sequence determined with SEQ ID NO: 1 and/or SEQ ID NO: 2 to identify differences in the sequence.

A method of diagnosing or prognosticating a body weight disorder in an animal comprising determining the level of neuronatin in a biological sample using an antibody to neuronatin, and using the measurement to evaluate the state of the animal.

An antibody to 1) a polypeptide according to SEQ ID NO: 3 (α-neuronatin) or SEQ ID NO: 4 (β-neuronatin), 2) a variant of one of the said sequences having at least 50% identity therewith, 3) a fragment of SEQ ID NO: 3 or SEQ ID NO: 4 comprising at least 7 amino acids, or 4) a variant of the said fragment having at least 80% identity therewith.

A method of identifying an interaction partner to neuronatin comprising using 1) a polypeptide according to SEQ ID NO: 3 (α-neuronatin) or SEQ ID NO: 4 (1-neuronatin), 2) a variant of one of the said sequences having at least 50% identity therewith, 3) a fragment of SEQ ID NO: 3 or SEQ ID NO: 4 comprising at least 7 amino acids, or 4) a variant of the said fragment having at least 80% identity therewith, to screen an expression library for interaction partners.

A method of identifying an interaction partner to neuronatin comprising using 1) a polynucleotide according to SEQ ID NO: 1 (α-neuronatin) or SEQ ID NO: 2 (β-neuronatin), 2) a variant of one of the said sequences having at least 30% identity therewith, 3) a fragment of SEQ ID NO: 1 or SEQ ID NO: 2 comprising at least 21 nucleotides, 4) a variant of one of the said fragments having at least 60% identity therewith, or 5) a sequence, which hybridises with SEQ ID NO: 1 or SEQ ID NO: 2 under highly stringent conditions, to screen an expression library for interaction partners.

A vector construct comprising 1) a polynucleotide according to SEQ ID NO: 1 (α-neuronatin) or SEQ ID NO: 2 (β-neuronatin), 2) a variant of one of the said sequences having at least 30% identity therewith, 3) a fragment of SEQ ID NO: 1 or SEQ ID NO: 2 comprising at least 21 nucleotides, 4) a variant of one of the said fragments having at least 60% identity therewith, or 5) a sequence, which hybridises with SEQ ID NO: 1 or SEQ ID NO: 2 under highly stringent conditions, and a promoter operably linked to the polynucleotide.

A packaging cell line capable of producing an infective virion comprising the vector of the invention.

A recombinant host cell comprising 1) a polynucleotide according to SEQ ID NO: 1 α-neuronatin) or SEQ ID NO: 2 (β-neuronatin), 2) a variant of one of the said sequences having at least 30% identity therewith, 3) a fragment of SEQ ID NO: 1 or SEQ ID NO: 2 comprising at least 21 nucleotides, 4) a variant of one of the said fragments having at least 60% identity therewith, or 5) a sequence, which hybridises with SEQ ID NO: 1 or SEQ ID NO: 2 under highly stringent conditions, or the vector of the invention.

A pharmaceutical composition comprising 1) a polynucleotide according to SEQ ID NO: 1 (α-neuronatin) or SEQ ID NO: 2 (β-neuronatin), 2) a variant of one of the said sequences having at least 30% identity therewith, 3) a fragment of SEQ ID NO: 1 or SEQ ID NO: 2 comprising at least 21 nucleotides, 4) a variant of one of the said fragments having at least 60% identity therewith, or 5) a sequence, which hybridises with SEQ ID NO: 1 or SEQ ID NO: 2 under highly stringent conditions, the vector of the invention, the packaging cell line of the invention or the host cell of the invention.

A method of preventing or treating a metabolic disorder in an animal comprising administering to the animal a 1) a polynucleotide according to SEQ ID NO: 1 (α-neuronatin) or SEQ ID NO: 2 (β-neuronatin), 2) a variant of one of the said sequences having at least 30% identity therewith, 3) a fragment of SEQ ID NO: 1 or SEQ ID NO: 2 comprising at least 21 nucleotides, 4) a variant of one of the said fragments having at least 60% identity therewith, or 5) a sequence, which hybridises with SEQ ID NO: 1 or SEQ ID NO: 2 under highly stringent conditions, the vector of the invention, the packaging cell line of the invention or the host cell of the invention.

A method of effecting an increase in locomotor activity and/or a reduction in food intake in an animal comprising administering to the animal an effective amount of 1) a polypeptide according to SEQ ID NO: 3 (α-neuronatin) or SEQ ID NO: 4 (β-neuronatin), 2) a biologically active variant of one of the said sequences having at least 50% identity therewith, 3) a biologically active fragment of SEQ ID NO: 3 or SEQ ID NO: 4 comprising at least 7 amino acids, or 4) a biologically active variant of the said fragment having at least 80% identity therewith.

As will appear from the Examples, the behavioural effects of treatment with the polypeptide of the invention are at least an increase in locomotor activity and/or a reduction in food intake. It is believed that these behavioural effects are coupled to the therapeutic effects in preventing or treating metabolic disorders of the polypeptide of the invention.

In its broadest aspect, the present invention relates to neuronatin proteins, isoforms or degenerate variants thereof, that demonstrate the ability to participate in the control of metabolic disorders, in particular mammalian body weight disorders and/or NIDDM. Data presented herein indicates that the mRNA product of the gene in question is regulated in ob/ob and db/db mice in the hypothalamus and special areas of the hypothalamus. The ob and db genotype mice lack a functional leptin and leptin receptor respectively and are important model systems for the study of obesity and diabetes, because these mice are hyperphagic and obese.

In addition, the Examples herein show that the neuronatin mRNA is specifically regulated in areas of the hypothalamus important in control of appetite and feeding behaviour in both ob/ob mice and fa/fa (leptin receptor deficient) rats. Also, the Examples show that neuronatin mRNA levels in specific hypothalamic areas is upregulated in ob/ob mice in response to daily injections of recombinant leptin. Finally, the Examples show that the neuronatin mRNA is co-regulated in response to daily injections of recombinant leptin in mice, which dramatically reduces the body mass of ob/ob mice and significantly affects the body weight of wild-type mice. Furthermore, we show that the Neuronatin gene is specific modulated in specific areas of the hypothalamus that is important for appetite and feeding behaviour.

In a first aspect, the modulators of the present invention comprise neuronatin polypeptides themselves serving as modulators of weight control as hereinafter defined, or conserved variants or fragments thereof, particularly such fragments lacking the signal peptide (alternatively referred to herein as mature neuronatin polypeptide), which polypeptides possess amino acid sequences such as set forth in SEQ ID NOS: 3-4).

In yet a further aspect, the present invention relates to antibodies that bind to the neuronatin polypeptide. Such antibodies may be generated against the full-length polypeptide, or antigenic fragments thereof. In one aspect, such antibodies inhibit the functional (i.e. body weight and fat composition modulating) activity of the neuronatin polypeptide. In another aspect, antibodies can be used to determine the level of circulating neuronatin polypeptide in plasma, serum or cerebrospinal fluid.

All of the foregoing materials are to be considered herein as modulators of body weight and fat composition, and as such, may be used in a variety of contexts. Specifically, the invention contemplates both diagnostic and therapeutic applications, including both nucleic acid molecules and peptides. Moreover, the modulation of body weight carries specific therapeutic implications and benefits, in that conditions where either obesity or, NIDDM represent undesired bodily conditions, can be remedied by the administration of one or more of the modulators of the present invention.

Thus, a method for modulating body weight of a mammal is proposed that comprises controlling the expression of the protein encoded by a nucleic acid having a nucleotide sequence selected from the sequences of SEQ ID NOS: 1-2 and degenerate and allelic variants thereof. Such control may be affected by the introduction of the nucleotides in question by gene therapy into cells of the patient or host to control or reduce obesity.

Correspondingly, the proteins defined by SEQ ID NOS: 3-4, conserved variants, active fragments thereof, and cognate small molecules could be formulated for direct administration for therapeutic purposes, to effect reduction or control of excessive body fat or weight gain. Correspondingly, antibodies and other antagonists to the stated protein materials, such as fragments thereof, could be prepared and similarly administered to achieve the converse effect. Accordingly, the invention is advantageously directed to a pharmaceutical composition comprising a neuronatin polypeptide of the invention, or alternatively an antagonist thereof, in an admixture with a pharmaceutically acceptable carrier or excipient.

The diagnostic uses of the present nucleotides and corresponding peptides extend to the use of the nucleic acids to identify further mutations of allelic variations thereof, so as to develop a repertoire of active nucleotide materials useful in both diagnostic and therapeutic applications. In particular, both homozygous and heterozygous mutations of the nucleotides in question could be identified that would be postulated to more precisely quantify the condition of patients, to determine the at-risk potential of individuals with regard to obesity. Corresponding DNA testing could then be conducted utilizing the aforementioned ascertained materials as benchmarks, to facilitate an accurate long term prognosis for particular tendencies, so as to be able to prescribe changes in either dietary or other personal habits, or direct therapeutic intervention, to avert such conditions.

Yet further, the present invention contemplates not only the identification of the nucleotides and corresponding proteins presented herein, but also the elucidation of the receptor to such materials. In such context, the polypeptides of SEQ ID NOS: 3-4 could be prepared and utilized to screen an appropriate expression library to isolate active receptors. The receptor could thereafter be cloned, and the receptor alone or in conjunction with the ligand could thereafter be utilized to screen for small molecules that may possess like activity to the modulators herein.

Yet further, the present invention relates to pharmaceutical compositions that include certain of the modulators hereof, preferably the polypeptides whose sequences are presented in SEQ ID NOS: 3-4, their antibodies, corresponding small molecule agonists or antagonists thereof, or active fragments prepared in formulations for a variety of modes of administration, where such therapy is appropriate. Such formulations would include pharmaceutically acceptable carriers, or other adjuvants as needed, and would be prepared in effective dosage ranges to be determined by the clinician or the physician in each instance.

Accordingly, it is a principal object of the present invention to provide modulators of body weight as defined herein in purified form, that exhibit certain characteristics and activities associated with control and variation of adiposity and fat content of mammals.

It is a further object of the present invention to provide methods for the detection and measurement of the modulators of weight control as set forth herein, as a means of the effective diagnosis and monitoring of pathological conditions wherein the variation in level of such modulators is or may be a characterizing feature.

It is a still further object of the present invention to provide a method and associated assay system for the screening of substances, such as drugs, agents and the like, that are potentially effective to either mimic or inhibit the activity of the modulators of the invention in mammals.

It is a still further object of the present invention to provide a method for the treatment of mammals to control body weight and fat content in mammals, and/or to treat certain of the pathological conditions of which abnormal depression or elevation of body weight is a characterizing feature.

It is a still further object of the present invention to prepare genetic constructs for use in genetic therapeutic protocols and/or pharmaceutical compositions for comparable therapeutic methods, which comprise or are based upon one or more of the modulators, binding partners, or agents that may control their production, or that may mimic or antagonize their activities.

Other objects of the invention will be apparent to the person skilled in the art from the following detailed description and examples.

### DETAILED DISCLOSURE OF THE INVENTION

The present invention relates to the elucidation of a protein, termed herein neuronatin polypeptide, isoforms thereof, and including degenerate variations thereof which demonstrates the ability to participate in the control of mammalian body weight, adiposity and NIDDM. Data presented herein indicates that the mRNA product of the gene in question is regulated in ob/ob, db/db mice in the hypothalamus and special areas of the hypothalamus in ob/ob mice and fa/fa rats. The ob/ob mice have a mutated leptin gene, whereas db/db mice and fa/fa rats lack a functional leptin receptor. All are important model systems for the study of obesity and diabetes, because they exhibit marked hyperphagia and obesity. Previous studies has revealed that the neuronatin mRNA exists in two alternative spliced forms (α, β), in which an 81-bp sequence in the middle coding region is present in the α-form, but not in the β-form [Joseph R, Dou D and Tsang W: Neuronatin mRNA: alternatively spliced forms of a novel brain-specific mammalian developmental gene; Brain Res. 1995 690 92-98].

In addition, the examples herein shows that the neuronatin mRNA is co-regulated in response to daily injections of recombinant leptin in mice which dramatically reduces the body mass of ob/ob mice and significantly affects the body weight of wild-type. Furthermore we show that the neuronatin gene is specific modulated in specific areas of the hypothalamus that is important for appetite and feeding behaviour.

In its primary aspect, the present invention is directed to the identification of materials that function as modulators of mammalian body weight. Weight control modulator peptides or their binding partners or other ligands or agents exhibiting either mimicry or antagonism to them or control over their production, may be prepared in pharmaceutical compositions, with a suitable carrier and at a strength effective for administration by various means to a patient experiencing abnormal fluctuations in body weight or adiposity, either alone or as part of an adverse medical condition such as cancer or AIDS, for the treatment thereof. A variety of administrative techniques may be utilized, among them oral administration, nasal and other forms of transmucosal administration, parenteral techniques such as subcutaneous, intravenous and intraperitoneal injections, catheterizations and the like. Average quantities of the recognition factors or their subunits may vary and in particular should be based upon the recommendations and prescription of a qualified physician or veterinarian.

In accordance with the above, an assay system for screening potential drugs effective to mimic or antagonize the activity of the weight modulator may be prepared. The weight modulator may be introduced into a test system, and the prospective drug may also be introduced into the resulting cell culture, and the culture thereafter examined to observe any changes in the activity of the cells, due either to the addition of the prospective drug alone, or due to the effect of added quantities of the known weight modulator.

To the extent that one or more of the modulators of the invention are secreted molecules, they can be used biochemically to isolate their receptor using the technology of expression cloning. It is important to note here that the term receptor here is used in its broadest form as any protein that is further activated by neuronatin binding/interaction. As neuronatin has been proposed to possess homology to the proteolipid class of proteins [Dou D and Joseph R. Cloning of human neuronatin gene and its localization to chromosome-20q 11.2-12: the deduced protein is a novel “proteolipid”; Brain Res. 1996 723 8-22], it is conceivable that neuronatin might function as a regulatory subunit of ion channels.

The discussion that follows with specific reference to the neuronatin gene bears general applicability to the class of modulators that comprise a part of the present invention, and is therefore to be accorded such latitude and scope of interpretation.

The functional activity of the neuronatin polypeptide can be evaluated transgenically with respect to feeding behaviour and NIDDM. In this respect, a transgenic mouse model can be used. The Neuronatin gene can be used in complementation studies employing transgenic mice. Transgenic vectors, including viral vectors, or cosmid clones (or phage clones) corresponding to the wild-type locus of candidate gene, can be constructed using the isolated neuronatin gene.

Alternatively, neuronatin genes can be tested by examining their phenotypic effect when expressed in antisense orientation in wild-type animals. In this approach, expression of the wild-type allele is suppressed, which leads to a mutant phenotype. RNA-RNA duplex formation (antisense-sense) prevents normal handling of mRNA, resulting in partial or complete elimination of wild-type gene effect. An important advantage of this approach is that only a small portion of the gene need be expressed for effective inhibition of expression of the entire cognate mRNA. The antisense transgene will be placed under control of its own promoter or another promoter expressed in the correct cell type, and placed upstream of the SV40 polyA site. This transgene will be used to make transgenic mice.

In the long term, the elucidation of the biochemical function of the neuronatin gene product (the neuronatin polypeptide or protein) is useful for identifying small molecule agonists and antagonists that affect its activity.

Various terms used throughout this specification shall have the definitions set out herein, for example, below.

The term “body weight modulator”, “modulator”, “modulators”, and any variants not specifically listed, may be used herein interchangeably, and as used throughout the present application refers to sequences defined in the claims. Accordingly, nucleotides displaying substantially equivalent or altered activity are likewise contemplated, including substantially homologous variants and allelic variations. Likewise, proteins displaying substantially equivalent or altered activity, including proteins modified deliberately, as for example, by site-directed mutagenesis, or accidentally through mutations in hosts that produce the modulators are likewise contemplated.

A composition comprising “A” (where “A” is a single protein, DNA molecule, vector, recombinant host cell, etc.) is substantially free of “B” (where “B” comprises one or more contaminating proteins, DNA molecules, vectors, etc. but excluding racemic forms of A) when at least about 75% by weight of the proteins, DNA, vectors (depending on the category of species to which A and B belong) in the composition is “A”. Preferably, “A” comprises at least about 90% by weight of the A+B species in the composition, most preferably at least about 99% by weight. It is also preferred that a composition, which is substantially free of contamination, contain only a single molecular weight species having the activity or characteristic of the species of interest.

The Neuronatin Polypeptides

The terms “protein”, which refers to the naturally occurring polypeptide, and “polypeptide” are used herein interchangeably with respect to the neuronatin gene product and variants thereof. The term “mature protein” or “mature polypeptide” particularly refers to the neuronatin gene product with the signal sequence (or a fusion protein partner) removed. As noted above, in specific embodiments neuronatin polypeptides of the invention include those having the amino acid sequences set forth herein e.g. SEQ ID NOS: 3-4, including the neuronatin polypeptide modified with conservative amino acid substitutions, as well as biologically active fragments, variants, and derivatives thereof. The term “biologically active,” is used herein to refer to a specific effect of the polypeptide, including but not limited to specific binding, e.g. to a receptor, antibody, or other recognition molecule; activation of signal transduction pathways on a molecular level; and/or induction (or inhibition by antagonists) of physiological effects mediated by the native neuronatin polypeptide in vivo. Neuronatin polypeptides, including fragments, variants, and derivatives, can be prepared synthetically, e.g. using the well known techniques of solid phase or solution phase peptide synthesis. Preferably, solid phase synthetic techniques are employed. Alternatively, neuronatin polypeptides of the invention can be prepared using well known genetic engineering techniques, as described infra. In yet another embodiment, the neuronatin polypeptide can be purified, e.g. by immunoaffinity purification, from a biological fluid, such as but not limited to plasma, serum, or urine, preferably human plasma, serum, or urine, and more preferably from a subject who overexpresses the polypeptide, such as an obese person suffering from a mutation in the neuronatin receptor or from obesity related to a mutation corresponding to “fatty.”

Fragments of the Neuronatin Polypeptide

In a particular embodiment, the present invention relates to fragments of SEQ ID NO: 3 and SEQ ID NO: 4. The said fragment comprises at least 9, preferably 11, more preferably 13, more preferably 15, more preferably 17, more preferably 19, more preferably 21, more preferably 23, more preferably 25, more preferably 27, more preferably 29, more preferably 31, more preferably 33, more preferably 35, more preferably 37, more preferably 39, more preferably 41, more preferably 43, more preferably 45, more preferably 47, more preferably 49 and most preferably 51 amino acids. In a preferred embodiment of the invention, the fragment of SEQ ID NO: 3 is the fragment consisting of amino acids 38-81, and the fragment of SEQ ID NO: 4 is the fragment consisting of amino acids 32-54.

Also, the present invention contemplates that natural occurring fragments of neuronatin exist and may be important. The protein sequence of neuronatin includes a number of sites that are frequently the target for proteolytic cleavage (see Example 2). It is possible that the full-length polypeptide may be cleaved at one or more such sites to form biologically active fragments. Such biologically active fragments may either agonize or antagonize the functional activity of the neuronatin polypeptide to reduce body weight.

The invention contemplates providing a neuronatin fragment having the minimum amino acid sequence necessary for a biological activity. This can be readily determined, e.g. by testing the activity of fragments of neuronatin for the ability to bind to OB-specific antibodies, inhibit the activity of the native neuronatin polypeptide, or agonize the activity of the native neuronatin peptide.

Variants of the Neuronatin Polypeptide

Preferably, the variant of SEQ ID NO: 3 or SEQ ID NO: 4 has at least 60%, more preferably 70%, more preferably 80%, more preferably 90%, and most preferably 95% identity therewith. Correspondingly, the variant of the fragment of SEQ ID NO: 3 or SEQ ID NO: 4 according to the present invention preferably has at least 85%, more preferably 90% and most preferably 95% identity with the fragments. The variants of the present invention includes all natural occurring neuronatin variants from different animal species, including e.g. variants originating from mouse, rats, dogs, cats, monkeys and ruminants, such as cow and ox.

The present invention specifically contemplates preparation of variants of the neuronatin peptide, which are characterized by being capable of a biological activity of neuronatin polypeptide, e.g. of binding to a specific binding partner of neuronatin peptide, such as a neuronatin receptor. In one embodiment, the variant agonizes neuronatin activity, i.e. it functions similarly to the neuronatin peptide. Preferably, a neuronatin agonist is more effective than the native protein. For example, a neuronatin agonist variant may bind to a neuronatin receptor with higher affinity, or demonstrate a longer half-life in vivo, or both. Nevertheless, neuronatin peptide agonist variants that are less effective than the native protein are also contemplated. In another embodiment, the variant antagonizes neuronatin activity. For example, a neuronatin variant that binds to a neuronatin receptor but does not induce signal transduction can competitively inhibit binding of native neuronatin to a receptor, thus decreasing neuronatin activity in vivo. Such a neuronatin antagonist variant may also demonstrate different properties from Neuronatin peptide, e.g. longer (or shorter) half-life in vivo, greater (or lesser) binding affinity for a neuronatin receptor, or both.

In one embodiment, a variant of neuronatin peptide is the neuronatin peptide modified by substitution of amino acids at positions on the polypeptide that are not essential for structure or function. For example, since the rat and human neuronatin peptide deviated from its other at only two positions (pos 62 and 77 in the β-neuronatin), substitution of divergent amino acid residues in the human sequence as compared to the rat amino acid sequence will likely yield useful variants of neuronatin peptide. For example, the Tyrosine residue at position 62 or the glutamine residue at position 77, or both from human may be substituted, e.g. with glycine, alanine, valine, cysteine, methionine, or threonine.

In another embodiment, a variant or fragment of the neuronatin polypeptide, preferably the human neuronatin polypeptide, is a truncated form of the polypeptide. It is possible that both the neuronatin α and β form is proteolytically processed (see Example 2). Thus, the active neuronatin a fragment consisting of amino acids number X through Y may be described by the following formula (assuming maximal two proteolytic sites): 78>X>4 and 81>Y>X. Likewise, the β neuronatin consisting of amino acids number X through Y may be described by the formula: 50>X>4 and 54>Y>X.

Variants of the Neuronatin Polynucleotide

Preferably, the variant of SEQ ID NO: 1 or SEQ ID NO: 2 has at least 40%, more preferably 50%, more preferably 60%, more preferably 70%, more preferably 80%, and most preferably 90% identity therewith. The variant of the fragment of SEQ ID NO: 1 or SEQ ID NO: 2 preferably has at least 60%, more preferably 70%, more preferably 80% and most preferably 50%, identity with said fragment.

Small Molecule Analogs and Peptidomimetics of Neuronatin Polypeptide

The structure of the neuronatin polypeptide, preferably human neuronatin polypeptide, can be analysed by various methods known in the art. The protein sequence can be characterized by a hydrophilicity analysis. A hydrophilicity profile can be used to identify the hydrophobic and hydrophilic regions of the neuronatin polypeptide, which may indicate regions buried in the interior of the folded polypeptide, and regions accessible on the exterior of the polypeptide. In addition, secondary structural analysis can also be done, to identify regions of neuronatin polypeptide that assume specific secondary structures. Manipulation of the predicted or determined structure, including secondary structure prediction, can be accomplished using computer software programs available in the art. The neuronatin peptide sequence may analysed by programs which predict cleavage of signal peptide to release mature peptide (see Example 2)

In yet a further embodiment, an analog of neuronatin polypeptide can be tested to determine whether it cross-reacts with an antibody specific for native neuronatin polypeptide, or specific fragments thereof. The degree of cross-reactivity provides information about structural homology or similarity of proteins, or about the accessibility of regions corresponding to portions of the polypeptide that were used to generate fragment-specific antibodies.

Screening for Neuronatin Analogs and Interaction Partners

Various screening techniques are known in the art for screening for analogs of polypeptides. Various libraries of chemicals are available. Accordingly, the present invention contemplates screening such libraries, e.g. libraries of synthetic compounds generated over years of research, libraries of natural compounds, and combinatorial libraries, as described in greater detail, infra, for analogs of neuronatin polypeptide. In one embodiment, the invention contemplates screening such libraries for compounds that bind to anti-neuronatin polypeptide antibodies, preferably anti-human neuronatin polypeptide antibodies. In another aspect, once the neuronatin receptor is identified, any screening technique known in the art can be used to screen for neuronatin receptor agonists or antagonists. The present invention contemplates screens for small molecule ligands or ligand analogs and mimics, as well as screens for natural ligands that bind to and agonize or antagonize activate neuronatin receptor in vivo.

Knowledge of the primary sequence of the receptor, and the similarity of that sequence with proteins of known function, can provide an initial clue as to the agonists or antagonists of the protein. Identification and screening of antagonists is further facilitated by determining structural features of the protein, e.g. using X-ray crystallography, neutron diffraction, nuclear magnetic resonance spectrometry, and other techniques for structure determination. These techniques provide for the rational design or identification of agonists and antagonists.

In another approach phage-display technologies can be used to isolate peptides, which bind neuronatin antibodies

In another aspect a two-hybrid screening system can be used to identify proteins and other peptides, which interact with the neuronatin peptide. One such two-hybrid method is a conventional two-hybrid method comprising cloning 1) a polynucleotide according to SEQ ID NO: 1 (α-neuronatin) or SEQ ID NO: 2 (β-neuronatin), 2) a variant of one of the said sequences having at least 30% identity therewith, 3) a fragment of SEQ ID NO: 1 or SEQ ID NO: 2 comprising at least 21 nucleotides, 4) a variant of one of the said fragments having at least 60% identity therewith, or 5) a sequence, which hybridises with SEQ ID NO: 1 or SEQ ID NO: 2 under highly stringent conditions, into a vector, expressing the said polynucleotide as a hybrid protein, the second part of the hybrid protein being a DNA-binding or a transcription activating domain of a heterologous protein, such as GAL4 or LEX, and using the hybrid protein to screen for possible interaction partners expressed as library fusion proteins, the result being the transcriptional activation of a appropriate reporter gene.

In another aspect, synthetic libraries such those described in a recent review [Dolle R E: Comprehensive survey of combinatorial libraries with undisclosed biological activity: 1992-1997; Mol. Divers. 1998 4 233-256] and the like can be used to screen for neuronatin receptor ligands according to the present invention. With such libraries, receptor antagonists can be detected using cells that express the receptor without actually cloning the neuronatin receptor.

Alternatively, assays for binding of soluble ligand to cells that express recombinant forms of the neuronatin receptor ligand-binding domain can be performed. The soluble ligands can be provided readily as recombinant or synthetic neuronatin polypeptide.

The screening can be performed with recombinant cells that express the neuronatin receptor, or alternatively, using purified receptor protein, e.g. produced recombinantly, as described above. For example, the ability of labelled, soluble or solubilised neuronatin receptor that includes the ligand-binding portion of the molecule, to bind ligand can be used to screen libraries, as described in the foregoing references.

Derivatives of Neuronatin Polypeptides

Generally, the present protein (herein the term “protein” is used to include “polypeptide,” unless otherwise indicated) may be derivatized by the attachment of one or more chemical moieties to the protein moiety. The chemically modified derivatives may be further formulated for intraarterial, intraperitoneal, intramuscular, subcutaneous, intravenous, oral, nasal, rectal, buccal, sublingual, pulmonary, topical, transdermal, or other routes of administration. Chemical modification of biologically active proteins has been found to provide additional advantages under certain circumstances, such as increasing the stability and circulation time of the therapeutic protein and decreasing immunogenicity (See e.g. U.S. Pat. No. 4,179,337).

Chemical Moieties for Derivatization

The chemical moieties suitable for derivatization may be selected from among water-soluble polymers. The polymer selected should be water-soluble so that the protein to which it is attached does not precipitate in an aqueous environment, such as a physiological environment. Preferably, for therapeutic use of the end-product preparation, the polymer will be pharmaceutically acceptable. One skilled in the art will be able to select the desired polymer based on such considerations as whether the polymer/protein conjugate will be used therapeutically, and if so, the desired dosage, circulation time, resistance to proteolysis, and other considerations. For the present proteins and peptides, these may be ascertained using the assays provided herein.

Polymer Molecules

The water soluble polymer may be selected from the group consisting of, for example, polyethylene glycol, copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propylene glycol homopolymers, polypropylene oxide/ethylene oxide co-polymers, polyoxyethylated polyols and polyvinyl alcohol. Polyethylene glycol propionaldehyde may provide advantages in manufacturing due to its stability in water.

The polymer may be of any molecular weight, and may be branched or un-branched. For polyethylene glycol, the preferred molecular weight is between about 2 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g. the duration of sustained release desired, the effects, if any on biological activity, the ease in handling, the degree or lack of antigenicity and other known effects of the polyethylene glycol to a therapeutic protein or analog).

Polymer/Protein Ratio

The number of polymer molecules so attached may vary, and one skilled in the art will be able to ascertain the effect on function. One may mono-derivatize, or may provide for a di-, tri-, tetra- or some combination of derivatization, with the same or different chemical moieties (e.g. polymers, such as different weights of polyethylene glycols). The proportion of polymer molecules to protein (or peptide) molecules will vary, as will their concentrations in the reaction mixture. In general, the optimum ratio (in terms of efficiency of reaction in that there is no excess un-reacted protein or polymer) will be determined by factors such as the desired degree of derivatization (e.g. mono, di-, tri-, etc.), the molecular weight of the polymer selected, whether the polymer is branched or un-branched, and the reaction conditions.

Attachment of the Chemical Moiety to the Protein

The polyethylene glycol molecules (or other chemical moieties) should be attached to the protein with consideration of effects on functional or antigenic domains of the protein. There are a number of attachment methods available to those skilled in the art, e.g. EP 401384 incorporated herein by reference (coupling PEG to G-CSF). See for instance Malik et al. [Malik F, Delgado C, Knusli C, Irvine A E, Fisher D, and Francis G E: Polyethylene glycol (PEG)-modified granulocyte-macrophage colony-stimulating factor (GM-CSF) with conserved biological activity; Exp. Hematol. 1992 20 1028-1035] with respect to the pegylation of G-CSF using tresyl chloride. For example, polyethylene glycol may be covalently bound through amino acid residues via a reactive group, such as a free amino or carboxyl group. Reactive groups are those to which an activated polyethylene glycol molecule may be bound. The amino acid residues having a free amino group may include lysine residues and the N-terminal amino acid residues, those having a free carboxyl group may include aspartic acid residues, glutamic acid residues and the C-terminal amino acid residue. Sulfhydryl groups may also be used as a reactive group for attaching the polyethylene glycol molecule(s). Preferred for therapeutic purposes is attachment at an amino group, such as attachment at the N-terminus or lysine group. Attachment at residues important for receptor binding should be avoided if receptor binding is desired.

N-Terminally Chemically Modified Proteins

One may specifically desire N-terminally chemically modified protein. Using polyethylene glycol as an illustration of the present compositions, one may select from a variety of polyethylene glycol molecules (by molecular weight, branching, etc.), the proportion of polyethylene glycol molecules to protein (or peptide) molecules in the reaction mix, the type of pegylation reaction to be performed, and the method of obtaining the selected N-terminally pegylated protein. The method of obtaining the N-terminally pegylated preparation (i.e. separating this moiety from other mono-pegylated moieties if necessary) may be by purification of the N-terminally pegylated material from a population of pegylated protein molecules. Selective N-terminal chemical modification may be accomplished by reductive alkylation, which exploits differential reactivity of different types of primary amino groups (lysine versus the N-terminus) available for derivatization in a particular protein. Under the appropriate reaction conditions, substantially selective derivatization of the protein at the N-terminus with a carbonyl group containing polymer is achieved. For example, one may selectively N-terminally pegylate the protein by performing the reaction at a pH, which allows one to take advantage of the pKₐ differences between the E-amino groups of the lysine residues and that of the -amino group of the N-terminal residue of the protein. By such selective derivatization attachment of a water soluble polymer to a protein is controlled: the conjugation with the polymer takes place predominantly at the N-terminus of the protein and no significant modification of other reactive groups, such as the lysine side chain amino groups, occurs. Using reductive alkylation, the water-soluble polymer may be of the type described above, and should have a single reactive aldehyde for coupling to the protein. Polyethylene glycol propionic aldehyde, containing a single reactive aldehyde, may be used.

Non-Coding Nucleic Acids

The inhibitor of a polynucleotide according to SEQ ID NO:1 or SEQ ID NO: 2 may be an anti-sense polynucleotide. The present invention extends to the preparation of antisense nucleotides and ribozymes that may be used to interfere with the expression of the weight modulator proteins at the translational level. This approach utilizes antisense nucleic acid and ribozymes to block translation of a specific mRNA, either by masking that mRNA with an antisense nucleic acid, cleaving it with a ribozyme, or masking/cleaving it with a siRNA (short interfering RNA).

Antisense nucleic acids are DNA or RNA molecules that are complementary to at least a portion of a specific mRNA molecule. In the cell, they hybridise to that mRNA, forming a double-stranded molecule. The cell does not translate an mRNA complexed in this double-stranded form. Therefore, antisense nucleic acids interfere with the expression of mRNA into protein. Oligomers of about fifteen nucleotides and molecules that hybridise to the AUG initiation codon will be particularly efficient, since they are easy to synthesize and are likely to pose fewer problems than larger molecules when introducing them into weight modulator peptide-producing cells.

Ribozymes are RNA molecules possessing the ability to specifically cleave other single-stranded RNA molecules in a manner somewhat analogous to DNA restriction endonucleases. Ribozymes were discovered from the observation that certain mRNA's have the ability to excise their own introns. By modifying the nucleotide sequence of these RNA's, researchers have been able to engineer molecules that recognize specific nucleotide sequences in an RNA molecule and cleave it. Because they are sequence-specific, only mRNA's with particular sequences are inactivated.

Investigators have identified two types of ribozymes, Tetrahymena-type and “hammerhead”-type. Tetrahymena-type ribozymes recognize four-base sequences, while “hammerhead”-type recognize eleven- to eighteen-base sequences. The longer the recognition sequence, the more likely it is to occur exclusively in the target mRNA species. Therefore, hammerhead-type ribozymes are preferable to Tetrahymena-type ribozymes for inactivating a specific mRNA species, and eighteen base recognition sequences are preferable to shorter recognition sequences.

siRNA's (short interfering RNA's) are short (15-25 nt) double stranded RNA's that can be used to suppress the expression of virtually every gene. siRNA's act by a mechanism known as RNA interference via an ancient mechanism that is present in all eukaryotes except bakers yeast, see for instance Zamore, P. D., Ancient pathways programmed by small RNA's; Science, 2002 296 1265-1269. siRNA's are double-stranded RNA molecules having a length of 21-23 nucleotides and bearing two 3′ overhanging ends. Such synthetic RNA molecules are detected by an enzyme complex, the RNA-induced silencing complex (RISC), which contains an endoribonuclease that uses the sequence encoded by the antisense strand to search for and find complementary mRNA that is subsequently destroyed. Efficient mRNA destruction by siRNA's involves a siRNA amplification step in which the siRNA acts as primer (by binding to mRNA) for the RNA-dependent RNA polymerase.

The DNA sequences described herein may thus be used to prepare antisense molecules, siRNA and ribozymes that cleave mRNA's for weight modulator proteins and their ligands, thus inhibiting, expression of the neuronatin gene, and leading to increased weight gain and adiposity.

In another embodiment, short oligonucleotides complementary to the coding and complementary strands of the neuronatin nucleic acid, or to non-coding regions of the neuronatin gene 5′, 3′, or internal (intronic) to the coding region are provided by the present invention. Such nucleic acids are useful as probes, either as directly labelled oligonucleotide probes, or as primers for the polymerase chain reaction, for evaluating the presence of mutations in the neuronatin gene, or the level of expression of neuronatin mRNA. Preferably, the non-coding nucleic acids of the invention are from the human neuronatin gene.

Stimulators and Inhibitors of Neuronatin

The stimulator of the neuronatin polypeptide according to the invention may be a small molecule. The inhibitor of the neuronatin polypeptide according to the invention may be a small molecule or an antibody thereto as described in more detail below.

Antibodies to the Neuronatin Polypeptide

According to the invention, neuronatin polypeptide produced recombinantly or by chemical synthesis, and fragments or other derivatives or analogs thereof, including fusion proteins, may be used as an immunogen to generate antibodies that recognize the neuronatin polypeptide. Such antibodies include but are not limited to polyclonal, monoclonal, chimeric, single chain, Fab fragments, and a Fab expression library.

A molecule is “antigenic” when it is capable of specifically interacting with an antigen recognition molecule of the immune system, such as an immunoglobulin (antibody) or T cell antigen receptor. An antigenic polypeptide contains at least about 5, and preferably at least about 10, amino acids. An antigenic portion of a molecule can be that portion that is immunodominant for antibody or T cell receptor recognition, or it can be a portion used to generate an antibody to the molecule by conjugating the antigenic portion to a carrier molecule for immunization. A molecule that is antigenic needs not be itself immunogenic, i.e. capable of eliciting an immune response without a carrier.

An “antibody” is any immunoglobulin, including antibodies and fragments thereof, that binds a specific epitope. The term encompasses polyclonal, monoclonal, and chimeric antibodies, the last mentioned described in further detail in e.g. U.S. Pat. Nos. 4,816,397 and 4,816,567, as well as antigen binding portions of antibodies, including Fab, F(ab′)₂ and F(v) (including single chain antibodies). Accordingly, the phrase “antibody molecule” in its various grammatical forms as used herein contemplates both an intact immunoglobulin molecule and an immunologically active portion of an immunoglobulin molecule containing the antibody-combining site. An “antibody-combining site” is that structural portion of an antibody molecule comprised of heavy and light chain variable and hyper variable regions that specifically binds antigen.

Exemplary antibody molecules are intact immunoglobulin molecules, substantially intact immunoglobulin molecules and those portions of an immunoglobulin molecule that contains the paratope, including those portions known in the art as Fab, Fab′, F(ab′)₂ and F(v), which portions are preferred for use in the therapeutic methods described herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g. a bi-specific (chimeric) monoclonal antibody.

The term “adjuvant” refers to a compound or mixture that enhances the immune response to an antigen. An adjuvant can serve as a tissue depot that slowly releases the antigen and also as a lymphoid system activator that non-specifically enhances the immune response. Often, a primary challenge with an antigen alone, in the absence of an adjuvant, will fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and potentially useful human adjuvants such as BCG (bacille Calmette-Guetin) and Corynebacterium parvum. Preferably, the adjuvant is pharmaceutically acceptable.

Various procedures known in the art may be used for the production of polyclonal antibodies to neuronatin polypeptide, or fragment, derivative or analog thereof. For the production of antibodies, various host animals can be immunized by injection with the neuronatin polypeptide, or a derivative (e.g. a fragment or fusion protein) thereof, including but not limited to rabbits, mice, rats, sheep, goats, etc. In one embodiment, the neuronatin polypeptide or fragment thereof can be conjugated to an immunogenic carrier, e.g. bovine serum albumin (BSA) or keyhole limpet hemocyanin (KLH). Various adjuvants may be used to increase the immunological response, depending on the host species, including but not limited to Freund's (complete and incomplete), mineral gels such as aluminium hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward the neuronatin polypeptide, or fragment, analog, or derivative thereof, any technique that provides for the production of antibody molecules by continuous cell lines in culture may be used. These include but are not limited to the hybridoma technique originally developed by Kohler and Milstein [Kohler G and Milstein C: Continuous cultures of fused cells secreting antibody of predefined specificity; Nature 1975 256 495-497] as well as the trioma technique, the human B-cell hybridoma technique, and the EBV-hybridoma technique to produce human monoclonal antibodies. Immortal, antibody-producing cell lines can be created by techniques other than fusion, such as direct transformation of B lymphocytes with oncogenic DNA, or transfection with Epstein-Barr virus, see also U.S. Pat. Nos. 4,341,761; 4,399,121; 4,427,783; 4,444,887; 4,451,570; 4,466,917; 4,472,500; 4,491,632; and 4,493,890.

In an additional embodiment of the invention, monoclonal antibodies can be produced in germ-free animals utilizing recent technology (PCT/US90/02545). According to the invention, human antibodies may be used and can be obtained by using human hybridomas [Cote R J, Morrissey D M, Houghton A N, Beattie E J Jr., Oettgen H F, and Old L J Generation of human monoclonal antibodies reactive with cellular antigens; Proc. Natl. Acad. Sci. U.S.A. 1983 80 2026-2030] or by transforming human B cells with EBV virus in vitro. In fact, according to the invention, techniques developed for the production of “chimeric antibodies” by splicing the genes from a mouse antibody molecule specific for a neuronatin polypeptide together with genes from a human antibody molecule of appropriate biological activity can be used; such antibodies are within the scope of this invention. Such human or humanized chimeric antibodies are preferred for use in therapy of human diseases or disorders (described infra), since the human or humanized antibodies are much less likely than xenogenic antibodies to induce an immune response, in particular an allergic response, themselves.

According to the invention, techniques described for the production of single chain antibodies (U.S. Pat. No. 4,946,778) can be adapted to produce neuronatin polypeptide-specific single-chain antibodies. An additional embodiment of the invention utilizes the techniques described for the construction of Fab expression libraries [Huse W D, Sastry L, Iverson S A, Kang A S, Alting-Mees M, Burton D R, Benkovic S J, and Lerner R A: Generation of a large combinatorial library of the immunoglobulin repertoire in phage lambda; Science 1989 246 1275-1281] to allow rapid and easy identification of monoclonal Fab fragments with the desired specificity for an neuronatin polypeptide, or its derivatives, or analogs.

Antibody fragments, which contain the idiotype of the antibody molecule, can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the production of antibodies, screening for the desired antibody can be accomplished by techniques known in the art, e.g. radio immunoassay, ELISA (enzyme-linked immunosorbent assay), “sandwich” immunoassays, immunoradiometric assays, gel diffusion precipitin reactions, immunodiffusion assays, in situ immunoassays (using colloidal gold, enzyme or radioisotope labels, for example), Western blots, precipitation reactions, agglutination assays (e.g. gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labelled. Many means are known in the art for detecting binding in an immunoassay and are within the scope of the present invention. For example, to select antibodies, which recognize a specific epitope of a neuronatin polypeptide, one may assay generated hybridomas for a product, which binds to a neuronatin polypeptide fragment containing such epitope. For selection of an antibody specific to a neuronatin polypeptide from a particular species of animal, one can select on the basis of positive binding with neuronatin polypeptide expressed by or isolated from cells of that species of animal.

The foregoing antibodies can be used in methods known in the art relating to the localization and activity of the neuronatin polypeptide, e.g. for Western blotting, imaging neuronatin polypeptide in situ, measuring levels thereof in appropriate physiological samples, etc.

In a specific embodiment, antibodies that agonize or antagonize the activity of neuronatin polypeptide can be generated. Such antibodies can be tested using the assays described infra for identifying ligands.

In a specific embodiment, antibodies are developed by immunizing rabbits with synthetic peptides predicted by the protein sequence, or with recombinant proteins made using bacterial expression vectors. The choice of synthetic peptides is made after careful analysis of the predicted protein structure, as described above. In particular, peptide sequences between putative cleavage sites are chosen. Synthetic peptides are conjugated to a carrier such as KLH hemocyanin or BSA using carbodiimide and used in Freund's adjuvant to immunize rabbits. In order to prepare recombinant protein, a suitable expression vector containing a strong promoter like e.g. the cytomegalovirus (CMV) can be used to express the polypeptide. Alternatively, one can use only hydrophilic domains to generate the fusion protein. The expressed protein will be prepared in quantity and used to immunize rabbits in Freund's adjuvant.

In another specific embodiment, recombinant neuronatin polypeptide is used to immunize chickens, and the chicken anti-neuronatin antibodies are recovered from egg yolk, e.g. by affinity purification on an OB-column. Preferably, chickens used in immunization are kept under specific pathogen free (SPF) conditions.

In another embodiment, antibodies against leptin are generated in ob/Neuronatin mice, which lack circulating neuronatin protein, and thus are expected to be capable of generating an anti-neuronatin polypeptide response since they will not be tolerant to the polypeptide, and wild-type mice. Spleen cells from both groups of mice can be fused with myeloma cells to prepare hybridomas for monoclonal antibodies.

In yet another embodiment, recombinant neuronatin polypeptide is used to immunize rabbits, and the polyclonal antibodies are immuno-purified prior to further use. The purified antibodies are particularly useful for semi-quantitative assays, particularly for detecting the presence of circulating neuronatin polypeptide in serum or plasma.

Panels of monoclonal antibodies produced against modulator peptides can be screened for various properties; i.e. isotype, epitope, affinity, etc. Of particular interest are monoclonal antibodies that neutralize the activity of the modulator peptides. Such monoclonals can be readily identified in activity assays for the weight modulators. High affinity antibodies are also useful when immunoaffinity purification of native or recombinant modulator is possible.

Preferably, the ant-modulator antibody used in the diagnostic and therapeutic methods of this invention is an affinity-purified polyclonal antibody. More preferably, the antibody is a monoclonal antibody (mAb). In addition, it is preferable for the anti-modulator antibody molecules used herein be in the form of Fab, Fab′, F(ab′) or F(v) portions of whole antibody molecules.

Diagnostic Implications

The present invention also relates to a variety of diagnostic applications, including methods for detecting the presence of conditions and/or stimuli that impact upon abnormalities in body weight or adiposity, by reference to their ability to elicit the activities, which are mediated by the present weight modulators. As mentioned earlier, the weight modulator peptides can be used to produce antibodies to themselves by a variety of known techniques, and such antibodies could then be isolated and utilized as in tests for the presence of particular transcriptional activity in suspect target cells. Alternatively, the nucleic acids of the invention can be employed in diagnosis. The diagnostic utility of the present invention extends to methods for measuring the presence and extent of the modulators of the invention in cellular samples or biological extracts (or samples) taken from test subjects, so that both the nucleic acids (genomic DNA or mRNA) and/or the levels of protein in such test samples could be ascertained. Given that the modulated activity of the nucleotide and presence of the resulting protein reflect the capability of the subject to inhibit obesity, the physician reviewing such results in an obese subject would determine that a factor other than dysfunction with respect to the presence and activity of the nucleotides of the present invention is a cause of the obese condition. Conversely, depressed levels of the nucleotide and/or the expressed protein would suggest that such levels must be increased to treat such obese condition, and an appropriate therapeutic regimen could then be implemented.

Antibody-Based Diagnostics

As suggested earlier, a diagnostic method useful in the present invention comprises examining a cellular sample or medium by means of an assay including an effective amount of an antagonist to a modulator protein, such as an anti-modulator antibody, preferably an affinity-purified polyclonal antibody, and more preferably a mAb. In addition, it is preferable for the anti-modulator antibody molecules used herein be in the form of Fab, Fab′, F(ab′)₂ or F(v) portions or whole antibody molecules. As previously discussed, patients capable of benefiting from this method include those suffering from cancer, AIDS, obesity or other conditions where abnormal body weight is a characteristic or factor. Methods for isolating the modulator and inducing anti-modulator antibodies and for determining and optimising the ability of anti-modulator antibodies to assist in the examination of the target cells are all well known in the art.

Also, antibodies including both polyclonal and monoclonal antibodies, and drugs that modulate the production or activity of the weight control modulators and other recognition factors and/or their subunits may possess certain diagnostic applications and may for example, be utilized for the purpose of detecting and/or measuring conditions where abnormalities in body weight are or may be likely to develop. For example, the modulator peptides or their active fragments may be used to produce both polyclonal and monoclonal antibodies to themselves in a variety of cellular media, by known techniques, such as the hybridoma technique utilizing, for example, fused mouse spleen lymphocytes and myeloma cells. These techniques are described in detail below. Likewise, small molecules that mimic or antagonize the activity(ies) of the receptor recognition factors of the invention may be discovered or synthesized, and may be used in diagnostic and/or therapeutic protocols.

Therapeutic Implications

The expression “metabolic disorder” in connection with the present invention comprises 1) the disease complex called metabolic syndrome X, hypertension, a body weight disorder, diabetes, including non-insulin-dependent diabetes mellitus (NIDDM), coronary heart disease, muscolo-skeletal disease, arteriosclerosis, retinopathy, peripheral neuropathy and nephropathy; 2) the disease states hyperlipidaemia, hyperglycaemia, microalbuminuria, glucose intolerance, insulin insensitivity, beta cell dysfunction and hypercoagulability; and 3) any disease related to the said disease states.

The polypeptides, nucleic acids, and antibodies of the invention have significant therapeutic potential. Preferably, a therapeutically effective amount of such an agent is administered in a pharmaceutically acceptable carrier, diluent, or excipient.

The phrase “pharmaceutically acceptable” refers to molecular entities and compositions that are physiologically tolerable and do not typically produce an allergic or similarly untoward reaction, such as gastric upset, dizziness and the like, when administered to a human. Preferably, as used herein, the term “pharmaceutically acceptable” means approved by a regulatory agency of the federal or a state government or listed in the US Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or solution saline solutions and aqueous dextrose and glycerol solutions are preferably employed as carriers, particularly for injectable solutions.

The phrase “therapeutically effective amount” is used herein to mean an amount sufficient to reduce by at least about 15%, preferably by at least 50%, more preferably by at least 90%, and most preferably prevent, a clinically significant deficit in the activity, function and response of the host. Alternatively, a therapeutically effective amount is sufficient to cause an improvement in a clinically significant condition in the host.

Administration of recombinant neuronatin polypeptide results in weight loss, in particular, a decrease in fat tissue. Neuronatin polypeptide can be prepared using standard bacterial and/or mammalian expression vectors, synthetically, or purified from plasma or serum, all as stated in detail earlier herein. Alternatively, increased expression of native neuronatin polypeptide may be induced by homologous recombination techniques, as described supra.

Reduction of neuronatin polypeptide activity (by developing antagonists, inhibitors, use of neutralizing antibodies, or antisense molecules) should result in weight gain as might be desirable for the treatment of the weight loss associated with cancer, AIDS or anorexia nervosa. Modulation of neuronatin activity can be useful for reducing body weight (by increasing its activity) or increasing body weight (by decreasing its activity).

Polypeptide-Based Therapeutic Treatment

In the simplest analysis, the neuronatin gene determines body weight in mammals, in particular, mice and man. The neuronatin gene product, and, correspondingly, cognate molecules, may effect particular areas in the brain know to be involved in feeding. It is believed that the neuronatin polypeptide is itself a signalling molecule, i.e. a hormone.

The neuronatin polypeptide, or functionally active fragment thereof, or an antagonist thereof, can be administered oral or parenteral, preferably parenteral. Because metabolic homeostasis is a continuous process, controlled release administration of neuronatin polypeptide is preferred. For example, the polypeptide may be administered using intravenous infusion, an implantable osmotic pump, a transdermal patch, liposomes, intranasally, by lung inhalation, or other modes of administration. In yet another embodiment, a controlled release system can be placed in proximity of the therapeutic target, i.e. the brain, thus requiring only a fraction of the systemic dose. In another embodiment, the therapeutic compound can be delivered in a vesicle, in particular a liposome.

In a further aspect, recombinant cells that have been transformed with the neuronatin gene and that express high levels of the polypeptide can be transplanted in a subject in need of neuronatin polypeptide. Preferably autologous cells transformed with neuronatin are transplanted to avoid rejection; alternatively, technology is available to shield non-autologous cells that produce soluble factors within a polymer matrix that prevents immune recognition and rejection.

The neuronatin polypeptide can be delivered by intravenous, intraarterial, intraperitoneal, intramuscular, or subcutaneous routes of administration. Alternatively, the neuronatin polypeptide, properly formulated, can be administered by nasal or oral administration. A constant supply of neuronatin can be ensured by providing a therapeutically effective dose (i.e. a dose effective to induce metabolic changes in a subject) at the necessary intervals, e.g. daily, every 12 hours, etc. These parameters will depend on the severity of the disease condition being treated, other actions, such as diet modification, that are implemented, the weight, age, and sex of the subject, and other criteria, which can be readily determined according to standard good medical practice by those of skill in the art.

Pharmaceutical Compositions

In yet another aspect of the present invention, pharmaceutical compositions of the above are provided. Such pharmaceutical compositions may be for administration for injection, or for oral, pulmonary, nasal or other forms of administration. In general, pharmaceutical compositions comprising effective amounts of protein or derivative products of the invention together with pharmaceutically acceptable diluents, preservatives, solubilizers, emulsifiers, adjuvants and/or carriers, are comprehended by the invention. Such compositions include diluents of various buffer content (e.g. Tris-HCl, acetate, phosphate), pH and ionic strength; additives such as detergents and solubilizing agents (e.g. Tween 80, Polysorbate 80), anti-oxidants (e.g. ascorbic acid, sodium metabisulfite), preservatives (e.g. Thimersol, benzyl alcohol) and bulking substances (e.g. lactose, mannitol); incorporation of the material into particulate preparations of polymeric compounds such as polylacetic acid, polyglycolic acid, etc. or into liposomes. Hylauronic acid may also be used. Such compositions may influence the physical state, stability, rate of in vivo release, and rate of in vivo clearance of the present proteins and derivatives. The compositions may be prepared in liquid form, or may be in dried powder, such as lyophilised form.

Administration of Compositions

Administration may be via any route known to be effective by the physician of ordinary skill, except that parenteral administration directly into the central nervous system is not a route taught or claimed in this invention. Peripheral, parenteral administration is preferred. Parenteral administration is commonly understood in the medical literature as the injection of a dosage form into the body by a sterile syringe or some other mechanical device such as an infusion pump. For the purpose of this invention, peripheral parenteral routes include intravenous, intramuscular, subcutaneous, and intraperitoneal routes of administration. Intravenous, intramuscular, and subcutaneous routes of administration of the compounds used in the present invention are more preferred. Intravenous and subcutaneous routes of administration of the compounds used in the present invention are yet more highly preferred. For parenteral administration, an active compound used in the present invention preferably is combined with distilled water at an appropriate pH.

Certain compounds used in the present invention to effect weight-loss may also be amenable to administration by the oral, rectal, nasal, or lower respiratory routes, which are non-parenteral routes. Of the said non-parenteral routes, the lower respiratory route is preferred for administration of peptides used in the instant invention. Various formulations of peptide compounds for administration by the lower respiratory tract are disclosed in U.S. Pat. Nos. 5,284,656 and 5,364,838. WO 96/19197 discloses aerosol formulations of various peptides suitable for enhancing lower respiratory tract absorption of the compounds used in the instant invention. The oral route of administration is preferred for compounds used in the instant invention.

Additional pharmaceutical methods may be employed to control the duration of action. Controlled release preparations may be achieved by the use of polymers to complex or absorb the active compound used in the present invention. Extended duration may be obtained by selecting appropriate macromolecules, for example, polyesters, polyamino acids, polyvinylpyrrolidone, ethylenevinyl acetate, methylcellulose, carboxymethylcellulose, or protamine sulphate, and by selecting the concentration of macromolecules, as well as the methods of incorporation, in order to prolong release. Another possible method to extend the duration of action by controlled release preparations is to incorporate an active compound used in the present invention into particles of a polymeric material such as polyesters, polyamino acids, hydrogels, poly (lactic acid) or ethylene vinylacetate copolymers. Alternatively, instead of incorporating a compound into these polymeric particles, it is possible to entrap a compound used in the present invention in microcapsules prepared, for example, by coacervation techniques or by interfacial polymerisation, for example, hydroxymethylcellulose or gelatin-microcapsules, respectively, or in colloidal drug delivery systems, for example, liposomes, albumin microspheres, microemulsions, nanoparticles, and nanocapsules, or in macroemulsions. Such teachings are known to those of skill in the art and disclosed, e.g. in Remington's Pharmaceutical Sciences, 1980.

Gene Therapy

The vector construct of the invention may be a viral vector, an adenoviral vector, an adenovirus-associated viral vector, a lentivirus vector, a retroviral vector or a vacciniaviral vector. The packaging cell line of the invention may be a phage. The recombinant host cell of the invention may be a human cell, a dog cell, a monkey cell, a rat cell or a mouse cell.

The Neuronatin Receptor

Development of small molecule agonists and antagonists of the neuronatin factor will be greatly facilitated by the isolation of its receptor. This can be accomplished by preparing active neuronatin polypeptide and using it to screen an expression library using standard methodology. Receptor binding in the expression library can be tested by administering recombinant polypeptide prepared using either bacterial or mammalian expression vectors, and observing the effects of short term and continuous administration of the recombinant polypeptide on the cells of the expression library, or by directly detecting binding of neuronatin polypeptide to the cells.

As it is presently believed that the neuronatin receptor is likely to be located in the hypothalamus, β-cells in the pancreas and perhaps adipocyte cells and liver, preferably cDNA libraries from these tissues will be constructed in standard expression cloning vectors. These cDNA clones would next be introduced into COS cells as pools and the resulting transformants would be screened with active ligand to identify COS cells expressing the neuronatin receptor. Positive clones can then be isolated so as to recover the cloned receptor. The cloned receptor would be used in conjunction with the neuronatin ligand (assuming it is a hormone) to develop the necessary components for screening of small molecules binding to the receptor.

A particular assay system that is to be utilized in accordance with the present invention is known as a receptor assay. In a receptor assay, the material to be assayed is appropriately labelled and then certain cellular test colonies are inoculated with a quantity of both the labelled and unlabeled material after which binding studies are conducted to determine the extent to which the labelled material binds to the cell receptors. In this way, differences in affinity between materials can be ascertained.

Accordingly, a purified quantity of the weight modulator may be radio-labelled and combined, for example, with antibodies or other inhibitors thereto, after which binding studies would be carried out. Solutions would then be prepared that contain various quantities of labelled and unlabeled non-combined weight modulator, and cell samples would then be inoculated and thereafter incubated. The resulting cell monolayers are then washed, solubilised and then counted in a gamma counter for a length of time sufficient to yield a standard error of <5%. These data are then subjected to Scatchard analysis after which observations and conclusions regarding material activity can be drawn. While the foregoing is exemplary, it illustrates the manner in which a receptor assay may be performed and utilize, in the instance where the cellular binding ability of the assayed material may serve as a distinguishing characteristic. In turn, a receptor assay will be particularly useful in the identification of the specific receptors to the present modulators, such as the neuronatin receptor.

A further assay useful and contemplated in accordance with the present invention is known as a “cis/trans” assay. Briefly, this assay employs two genetic constructs, one of which is typically a plasmid that continually expresses a particular receptor of interest when transfected into an appropriate cell line, and the second of which is a plasmid that expresses a reporter such as luciferase, under the control of a receptor/ligand complex. Thus, for example, if it is desired to evaluate a compound as a ligand for a particular receptor, one of the plasmids would be a construct that results in expression of the receptor in the chosen cell line, while the second plasmid would possess a promoter linked to the luciferase gene in which the response element to the particular receptor is inserted. If the compound under test is an agonist for the receptor, the ligand will complex with the receptor, and the resulting complex will bind the response element and initiate transcription of the luciferase gene. The resulting chemiluminescence is then measured photometrically, and dose response curves are obtained and compared to those of known ligands. The foregoing protocol is described in detail in U.S. Pat. No. 4,981,784 and WO 88/03168, for which purpose the artisan is referred.

Once a recombinant cell-line which expresses the neuronatin receptor gene sequence is identified, the recombinant neuronatin receptor can be analysed. This is achieved by assays based on the physical or functional properties of the neuronatin receptor, including radioactive labelling of the receptor followed by analysis by gel electrophoresis, immunoassay, ligand binding, etc. Furthermore, antibodies to the neuronatin receptor could be generated as described above.

The structure of the neuronatin receptor can be analysed by various methods known in the art. Preferably, the structure of the various domains, particularly the neuronatin-binding site, is analysed. Structural analysis can be performed by identifying sequence similarity with other known proteins, particular hormone and protein receptors. The degree of similarity (or homology) can provide a basis for predicting structure and function of the neuronatin receptor, or a domain thereof. In a specific embodiment, sequence comparisons can be performed with sequences found in GenBank, using, for example, the FASTA and FASTP programs [Pearson W R and Lipman D J: Improved tools for biological sequence comparison; Proc. Natl. Acad. Sci. U.S.A. 1988 85 2444-2448].

The protein sequence can be further characterized by a hydrophilicity analysis. A hydrophilicity profile can be used to identify the hydrophobic and hydrophilic regions of the neuronatin receptor protein, which may in turn indicate extra-cytoplasmic, membrane binding, and intra-cytoplasmic regions.

Secondary structural analysis can also be done, to identify regions of the neuronatin receptor that assume specific secondary structures.

Manipulation, translation, and secondary structure prediction, as well as open reading frame prediction and plotting, can also be accomplished using computer software programs available in the art.

Identification and isolation of a gene encoding a neuronatin receptor of the invention provides for expression of the receptor in quantities greater than can be isolated from natural sources, or in indicator cells that are specially engineered to indicate the activity of a receptor expressed after transfection or transformation of the cells. Accordingly, in addition to rational design of agonists and antagonists based on the structure of the neuronatin polypeptide, the present invention contemplates an alternative method for identifying specific ligands of neuronatin receptor using various screening assays known in the art.

Definitions

In connection with the present invention the various expression stated below have the following meanings:

A “variant” of a polypeptide sequence means any natural occurring or synthetic mutant of the sequence including allelic variants, degenerative variants, isoforms, sequences comprising amino acid substitutions, insertions, deletions and truncations, and derivatives of the sequence, including derivates containing chemical modifications in the amino acid side chains.

A “variant” of a polynucleotide sequence means any natural occurring or synthetic mutant of the sequence including allelic variants, degenerative variants, isoforms, sequences comprising nucleotide substitutions, insertions, deletions and truncations, and derivatives of the sequence, including derivates containing chemical modifications.

The expression “highly stringent conditions” in connection with polynucleotide hybridisation means 1 M Na, a temperature of 65° C. and a incubation period of 24 hours.

The expression “animal” means any animal, wherein neuronatin or a variant form thereof is expressed.

### BRIEF DESCRIPTION OF THE DRAWINGS

The present invention is further illustrated by reference to the accompanying drawing, in which:

FIG. 1 shows an outline of the restriction fragment differential display (RFDD-PCR);

FIG. 2 shows an RFDD-PCR picture of differential neuronatin gene expression between wild type, ob/ob and db/db mice;

FIG. 3 shows the predicted signal-peptide and cleavage site in the neuronatin α-polypeptide; and

FIG. 4 shows the predicted signal-peptide and cleavage site in the neuronatin β-polypeptide.

FIG. 5 shows autoradiograms of rat (left column) and mice (right column) brain sections through the hypothalamus hybridized with a neuronatin cRNA sense (A and E) and antisense probe (B,C,D,F,G,H).

FIG. 6 shows neuronatin mRNA expression levels in the dorsomedial hypothalamic nucleus and the Arcuate nucleus in obese fa/fa and lean Fa/? rats.

FIG. 7 shows neuronatin mRNA expression levels in the dorsomedial hypothalamic nucleus and lateral hypothalamic as well as in the PVN of vehicle treated lean Ob/?, vehicle treated obese ob/ob and leptin treated obese ob/mice.

FIG. 8 shows neuronatin immunoreactive cells in the hypothalamic suprachiasmatic nucleus (A) and paraventricular nucleus (B). A and B is stained with antibody #333rb-4 diluted 1:1000 and C and D (which shows no staining) show the suprachiasmatic and paraventricular nucleus stained with pre-immune serum (diluted 1:1000) from rabbit 333rb).

FIG. 9 Western blot using polyclonal antibody (#333rb-4) raised against a 44aa neuronatin α peptide fragment.

FIG. 10 shows the acute effect of two doses (20 μg and 40 μg) of Neuronatin alfa (aa 38-81) and vehicle on food intake. Animals are injected just prior to lights out and food intake is measured every 15 minutes for a 15 hour period.

FIG. 11 shows the acute effect of two doses (20 μg and 40 μg) of Neuronatin alfa (aa 38-81) on locomotor behavior. Animals are injected just prior to lights out and locomotor behavior registered online with a subtotal every 15 minutes for a 15-hour period.

FIG. 12 shows the acute effect of a dose of 40 μg Neuronatin beta (aa 32-54) and vehicle on food intake. Animals are injected just prior to lights out and food intake is measured every 15 minutes for a 15-hour period.

FIG. 13 shows the acute effect of 40 μg Neuronatin beta (aa 32-54) on locomotor behavior. Animals are injected just prior to lights out and locomotor behavior (registered as two consecutive beam breaks) is recorded every 15 minutes for a 15-hour period.

FIG. 14 shows the body weight gain expressed as a percentage of body weight day 1 in rats treated twice daily (10 μl icv injections morning and evening) with 20 μg and 40 μg Neuronatin alfa (aa 38-81) and vehicle. Body weight gain is lower in Neuronatin alfa treated rats.

FIG. 15 shows the effects of administration twice daily (10 μl icv injections morning and evening) of 20 μl and 40 μl Neuronatin alfa (aa 38-81) and vehicle on daily food intake (cumulated food intake measured over 24 hours). No effect on food intake is observed.

### EXAMPLES

The invention is further illustrated with reference to the following examples, which are not intended to be in any way limiting to the scope of the invention as claimed.

### Example 1

Identification of Differentially Expressed Neuronatin from ob/ob and db/db Mice.

Animals

12 adult male C57BL/6J mice (6 weeks old; 22±3 g; Mean±SEM), db/db mice (6 weeks old 57±3 g; Mean±SEM), and ob/ob mice (6 weeks old; 57±2 g; Mean±SEM) obtained from Møllegåarden, LI. Skensved, Denmark) were used in the experiment (Table I). The animals were kept under standard laboratory conditions (12:12 h light/dark cycle; lights on at 06.00 h) in a humidity- and temperature controlled room with free access to standard food and water for another 6 weeks.

TABLE I
Groups in the present experiment

| Group | Genotype | Glucseconc./[mM] |
|---|---|---|
| Group 1 (n = 4) | C57BL/6J | 3.3 ± 1 |
| animal no. 1-3 | | |
| Group 2 (n = 4) | C57BL/6J:ob/ob | 10 ± 1 |
| animal no. 4-6 | | |
| Group 3 (n = 4) | C57BL/6J:db/db | 22 ± 10 |
| animal no. 7-9 | | |

Glucose Measurement and Isolation of Hypothalamus

At the day of the experiment, the animals were sacrificed 2-3 hours after the light on (t=0) and blood glucose is measured from trunk blood. Whole blood glucose (mM) concentration was analysed by the immobilised glucose oxidase method using a drop of blood (<5 ml; Elite autoanalyser, Bayer, Denmark) following the manufacturer's manual.

The hypothalamus was rapidly removed and frozen individually at dry ice. The tissue was kept at −80° C. until extraction.

RNA Extraction

The RNA was extracted using the phenol-chloroform extraction method of Chomczynski & Sacchi [Chomczynski P & Sacchi N: Single-step method of RNA isolation by acid guanidinium thiocyanate-phenol-chloroform extraction; Anal. Biochem. 1987 162 156-159] from the hypothalamus pooled from 4 animals originating from each group (C57BL, C57BL:ob/ob and C57BL:db/db respectively). The RNA was evaluated on a gel, and only RNA showing distinct bands on this gel was used for further analysis. Further, the RNA concentration was measured using OD.

Preparation of the Genetic Profile of Cardiomycyte mRNA Using “Restriction Fragment Differential Display”—PCR(RFDD-PCR).

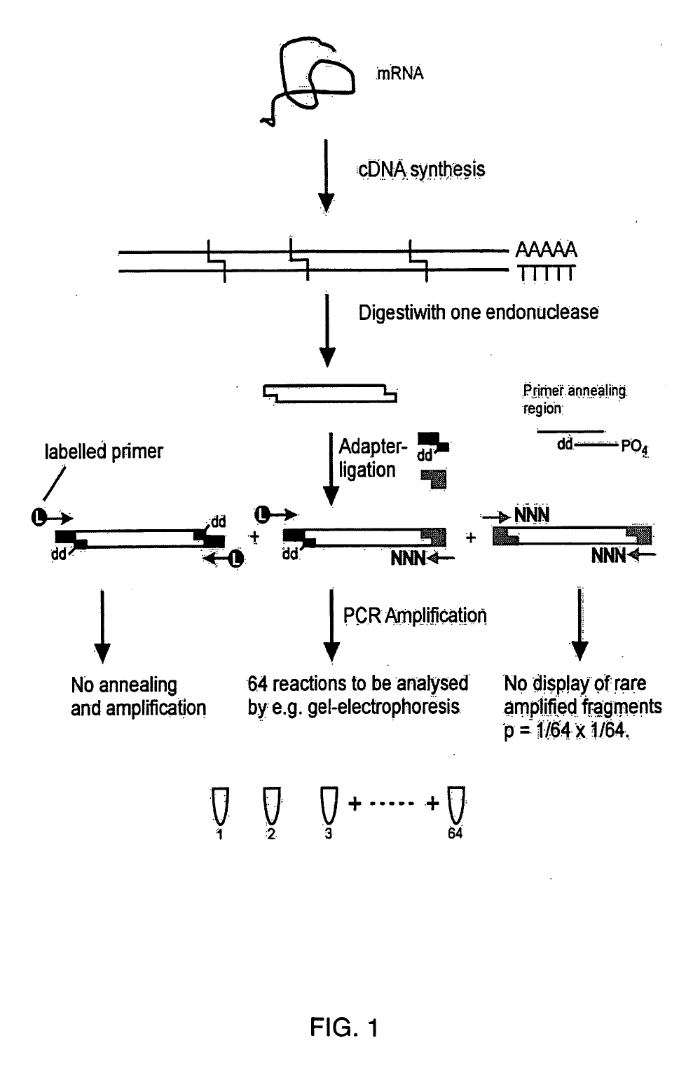

The procedure to generate the genetic profile of the RNA isolated from the liver of ob/ob and db/db mice is out-lined in FIG. 1. The RFDD-PCR technology is described in detail in PATENT PCT/DK98/00186 and will in the following briefly be described. Basically, RFDD-PCR correlates gene expression to the concentration of the corresponding cDNA amplified by PCR, as in traditional DD. However, the serious problems of false-positive results in DD are avoided in RFDD-PCR through modifications enabling use of high-stringency PCR conditions.

Synthesized double-stranded cDNA is digested with the TaqI restriction enzyme, which has a 4-base recognition sequence. Following digestion, two specially designed DNA adaptors are ligated to the cDNA fragments. One of the adaptors lacks a portion of the complementary strand and has a modification on the 3′end preventing the polymerase from filling-in this missing sequence. Use of this adaptor with the missing sequence and the extension protection group (EPG) is essential to the RFDD-PCR procedure. One of the primers used in the PCR reactions (the 0-extension primer) binds to the sequence missing from this EPG-adaptor. Thus, priming with the 0-extention primer does not occur until the binding site for this primer is synthesised when the complementary strand is synthesised during the first round of PCR. This approach allows the primer that binds to the other standard adaptor to determine which cDNA fragments are amplified in the reaction.

The other primer used in the PCR reactions, the 3-extension primer, binds to the regular adaptor and extends 3 bases (NNN) past the adaptor junction into the cDNA fragment. Since the O-extension primer cannot bind for the first round of PCR, it is these 3 bases that determine which cDNA are amplified in a specific reaction. Thus, each PCR reaction only amplifies a specific subset of cDNA fragments that have a specific three base sequence upstream of the TaqI restriction site. To amplify all variations, which provide a complete cross section of all the expressed sequences, only 64 (i.e. 43) different primers are necessary. Visualisation of the PCR products is obtained by labelling of the 0-extension primer.

We applied this approach to visualize and identify differentially expressed genes in the ob/ob and db/db genotype mice, which are models for obesity and NIDDM. The RNA was isolated from the liver of these animals and compared to wild type mice by RFDD-PCR analysis.

First-strand synthesis. 500 ng of DNase treated total RNA was reverse-transcribed in 25 μl volume using 100 units displayThermo (Display Systems Biotech), 0.75 μM T25V primer and 1 mM dNTP in cDNA buffer 1 (500 mM Tris HCl pH 8.3, 800 mM KCl, 100 mM MgCl₂, 40 mM DTT). The reaction was incubated at 42° C. for 2 hours.

Second-strand synthesis. To the 25 μl 1^{st} strand synthesis was added 2.5 μl 5 mM dNTP, 1.2 μl DNA Polymerase I (10 U/μl, display systems Biotech), 0.8 μl RNase H (1 U/μl), 7.5 μl 10×cDNA buffer 2 (350 mM Tris HCl pH 7.4, 40 mM MgCl2, 10 mM (NH₄)₂SO₄, 30 mM DTT) and distilled H₂O to a total of 75 μl. The reaction was incubated at 16° C. for 2 h. The reaction was subsequently phenol:chloroform extracted, precipitated by ethanol and dissolved in 20 μl H₂O. 10 μl cDNA was checked on an agarose gel for cDNA smear between 100 and 2000 bp.

Endonuclease digestion. To the remaining 10 μl cDNA was added 2.0 μl 10× displayPROFILE Buffer (100 mM TrisAc pH 7.5, 100 mM MgAc, 500 mM KAc), 0.5 μl TaqI Restriction enzyme (10 U/μl, display Systems Biotech), sterile H₂O until 20 μl. The reaction was incubated at 65° C. for 2 hours in a thermocycler with heated lid to avoid evaporation of the samples.

Ligation. To the 20 μl endonuclease digestion mix is added 0.75 μl 10× displayPROFILE Buffer, 0.75 μl 15 μM Adaptor mix (the adaptor mix contains both the standard adaptor and the adaptor containing the extension protection group), 1.25 μl 10 mM ATP, 0.30 μl T4 DNA ligase (1 U/μl, Display Systems Biotech) and sterile H₂O until 27.5 μl. The ligation reaction was incubated at 37° C. for 3 hours. (Note: The incubation is carried out at 37° C. and not the standard 16° C. to ensure that the TaqI-endonuclease is active during the ligation. This will ensure that there will be no re-ligation of the TaqI-fragments as the adaptors destroys the TaqI-site after ligation.)

Amplification of the Template

³³P end labelling of 0-extension primer. For the labelling of 0-extension primer for one PCR reaction was used 0.1 μl 10× displayPROFILE Buffer, 0.40 μl 0-extension Primer (10 μM), 0.20 μl [g33P]-ATP (3000 Ci/mmol or 25 mCi, ICN), 0.02 μl T4 Polynucleotide kinase (5 U/μl) and finally H₂O to 1.00 μl. The reaction was scaled up dependent on the number of PCR reaction used for the different 3-extension primers.

PCR amplification of the template. For one reaction was mixed 2.0 μl displayTAQ FL 10× Reaction Buffer, 0.8 μl 5 mM dNTP, 1.0 μl labelled 0-extension primer (4 μM), 0.3 μl displayTAQ FL (5 U/μl, Display Systems Biotech), 4.0 μl 3-extension primer (1 μM) and distilled H₂O to a final volume of 20.0 μl. For each of the 3 samples was performed 64 PCR reactions using the 64 different 3-extension primers. The following PCR-amplification profile was performed: precycle 94 C, 1 minuntes for the first 10 cycles (94° C., 30 sec; 60° C. with touchdown by 0.50 C for each cycle until 55° C. is reached, 72° C., 1 min), for the last 25 cycles (94° C., 30 sec; 55° C., 30 sec; 72° C., 1 min).

Isolation of modulated gene transcripts. Samples containing the PCR-fragments were applied to a standard 6% Polyacryl amide sequencing gel, separated by electrophoresis for 280 min and dried onto Whatman paper at 80° C.

Detection of the Down Regulated Neuronatin Gene in ob/ob and db/db Mice

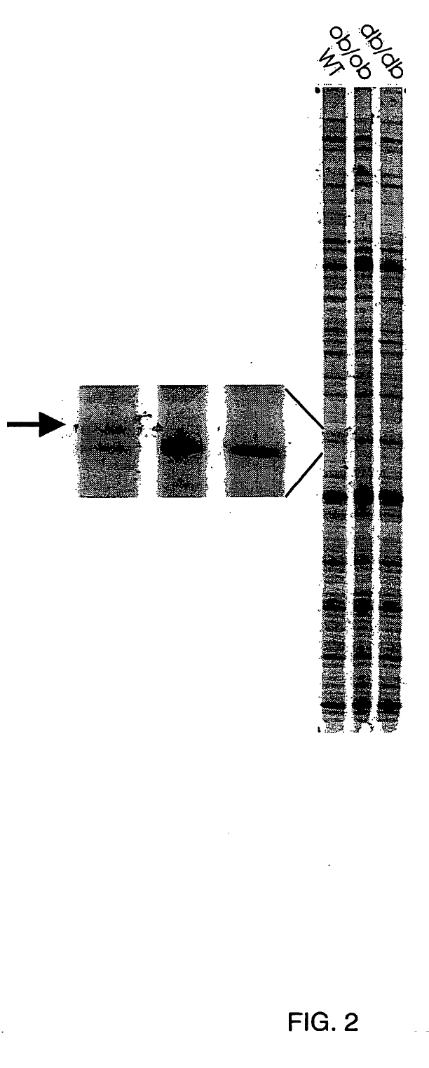

FIG. 2 shows an example of how neuronatin was identified as a gene involved in obesity. The genetic profile analysis (RFDD-PCR) was performed on 3 samples: 1) Control—mRNA isolated from liver of wild type mice, 2) mRNA isolated from liver of the ob/ob mice, 3) mRNA isolated from liver of the db/db genotype mice. This example constitutes 1/64 of the complete genetic profile. The neuronatin gene was clearly down regulated in the ob/ob and db/db mice.

Isolation of the Gene

Bands, found interesting, were lined up with markings on the film and the dried gel. The fragments were accordingly excised from the gel and dissolved in 50 μl TE buffer (10 mM Tris HCl ph 7.5, 1 mM EDTA). Following amplification using the same PCR conditions and primers as in the initial PCR the PCR fragments was cloned into a cloning vector and subsequently characterized by sequencing. The following sequence was obtained which shows 100% identity to the 3′-end of the neuronatin gene

5′GAGATATGGGCGTAGGCACCACATTCTGATCTGGACCAAGTCGGAA
CAGTACCATCTCAGCCGCACAAGATCCTACCATGAAGA

### Example 2

Prediction of Signal Peptide Sequences and Cleavage Sites in Neuronatin.

The neuronatin polypeptide was analysed on a neural network, which has been trained to predict signal peptides and cleavage sites in peptide sequences [Nielsen H, Engelbrecht J, Brunak S & von Heijne G: A neural network method for identification of prokaryotic and eukaryotic signal peptides and prediction of their cleavage sites; Int. J. Neural Syst. 1997 8 581-599]. The polypeptide can be submitted to such an analysis at the SignalP WWW server [http://www.cbs.dtu.dk/services/SignalP/] which will return three scores between 0 and 1 for each position in a peptide sequence:

C-Score (Raw Cleavage Site Score)

The output score from networks trained to recognize cleavage sites vs. other sequence positions. It is trained to be high at position +1 (immediately after the cleavage site) and low at all other positions.

S-Score (Signal Peptide Score)

The output score from networks trained to recognize signal peptide vs. non-signal-peptide positions. Trained to be high at all positions before the cleavage site and low at 30 positions after the cleavage site and in the N-terminals of non-secretory proteins.

Y-Score (Combined Cleavage Site Score)

The prediction of cleavage site location is optimised by observing where the C-score is high and the S-score changes from a high to a low value. The Y-score formalizes this by combining the height of the C-score with the slope of the S-score.

Specifically, the Y-score is a geometric average between the C-score and a smoothed derivative of the S-score (i.e. the difference between the mean S-score over d positions before and d positions after the current position, where d varies with the chosen network ensemble).

All three scores are averages of five networks trained on different partitions of the data.

For each sequence, SignalP will report the maximal C-, S-, and Y-scores, and the mean S-score between the N-terminal and the predicted cleavage site. These values are used to distinguish between signal peptides and non-signal peptides. If the sequence is predicted to have a signal peptide, the cleavage site is predicted to be immediately before the position with the maximal Y-score.

For a typical signal peptide, the C- and Y-scores will be high at position +1, while the S-score will be high before the cleavage site and low thereafter.

The analysis of neuronatin α gave the following results

<Is the sequence a signal peptide?

| # Measure | Position | Value | Cutoff | Conclusion |
|---|---|---|---|---|
| max. C | 38 | 0.435 | 0.37 | YES |
| max. Y | 38 | 0.533 | 0.34 | YES |
| max. S | 23 | 0.949 | 0.88 | YES |
| mean S | 1-37 | 0.803 | 0.48 | YES |

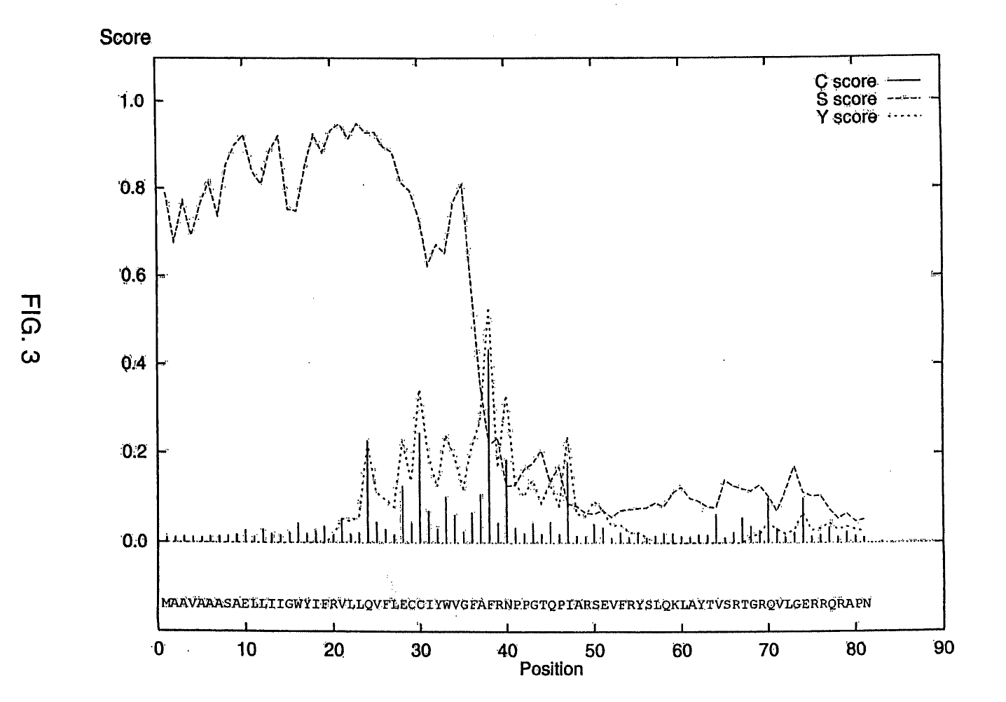

# Most likely cleavage site between pos. 37 and 38: GFA-FR See also FIG. 3

The analysis of neuronatin β gave the following results

<Is the sequence a signal peptide?

| # Measure | Position | Value | Cut-off | Conclusion |
|---|---|---|---|---|
| max. C | 32 | 0.275 | 0.37 | NO |
| max. Y | 32 | 0.358 | 0.34 | YES |
| max. S | 10 | 0.922 | 0.88 | YES |
| mean S | 1-31 | 0.695 | 0.48 | YES |

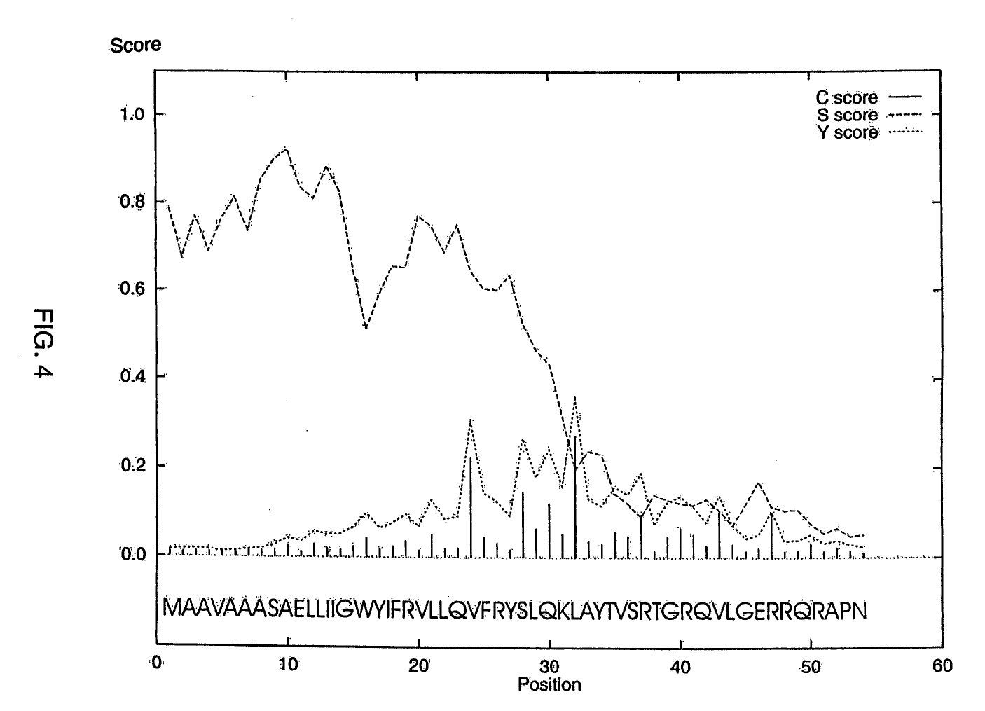

# Most likely cleavage site between pos. 31 and 32: SLQ-KL See also FIG. 4

Thus, the present analysis predicts that native neuronatin α and β fulfils the criteria of pre-protein where the first 30-40 amino acids is suggested to contain a signal peptide with probability. This signal peptide is predicted to be proteolytically removed whereby a physiological active peptide is generated.

### Example 3

Identification of Hypothalamic Areas in the Mouse and Rat Expressing Neuronatin mRNA

Cloning of 341 Base-Pair Neuronatin cDNA Fragment into Plasmid Vector:

Total RNA is extracted from frozen (−80° C.) hypothalami obtained from male C57BL/6J mice (Charles River, Sweden) using the RNeasy RNA purification kit (Qiagen, Maryland, USA). Integrity and concentration of the extracted RNA is evaluated on a gel. RT-PCR is performed in a total volume of 20 μl using 1 μg total RNA, 20 U of Superscript Reverse Transcriptase (GIBCO, Life Technologies), enzyme buffer (1×), 10 mM DTT, 2 pM/μl oligodT(15) primers, 1 mM dNTP's and 0.5 U/μl RNase inhibitor. To generate a neuronatin PCR fragment, 2 μl of the template cDNA reaction is mixed with primers directed against the murine neuronatin cDNA sequence (accession no: x83570; primers: 5′ ⁶⁷² CATCMCCAGCAGAATGGAC ⁶⁹¹, 3′ ¹⁰¹³ TGAGACCAGGGATMGCAAG ⁹⁹⁴). The final reaction mix (total volume 50 μl) contains 2 μl cDNA reaction, 1.5 mM MgCl2, 0.75 mM of each primer, 1.25 U Taq DNA polymerase (Sigma-Aldrich), 1× buffer, 0.2 mM dNTP's. The size of the generated PCR product is checked on a gel. The PCR product is cloned into pBluescript KS+. Subsequently E. coli DH5 alpha cells are transfected with the plasmid DNA and grown on Ampicillin/X-gal containing culture plates. The insert of a positive clone is analyzed by PCR and the PCR-product sequenced (sequencing by MWG-biotech, Germany). The obtained raw data sequence is analyzed using the BLAST search engine (http://www.ncbi.nlm.nih.gov/BLAST/) verifying a 100% identity to murine neuronatin cDNA.

Plasmid Purification, Linearization and In Vitro Transcription:

Plasmid containing E. coli are grown overnight at 37° C. in LB medium and plasmid DNA purified using the Qiagen Midi-Prep kit. For the in vitro transcription plasmid DNA is linearized using restriction enzymes (antisense: HindIII, sense: BamHI). S³⁵ labeled antisense (T7 polymerase) and sense (T3 polymeras) cRNA probes are prepared as follows: 1× transcription buffer, 10 mM DTT, RNase inhibitor (0.5 U/μl), CTP/ATP/GTP mix (1 mM), S³⁵-alfa-UTP (Amersham Pharmacia), linearized DNA (1 μg) and polymerase (T3 or T7, 40 U) are mixed and incubated for 2 hours at 37° C. Subsequently, DNA is digested by the addition of 1 μl RQ1 2 μl yeast tRNA and 1 μl RNase inhibitor (0.5 U). The transcripts are purified using Micro-Biospin 30 columns (Qiagen) followed by precipitation in 2.0M ammoniumacetate and ethanol. The transcripts are diluted in a 1:1 mixture of 100% de-ionized formamide and Tris (10 mM) EDTA (1 mM)-DTT (10 mM) buffer (pH 7.5). Specific activity of the generated transcripts is determined using a beta-counter.

In Situ Hybridization:

Two male Spraque Dawley rats (Charles River, Sweden) and two male C57BL/6J mice (Charles River, Sweden) are used. The animals are sacrificed by decapitation and brains removed and frozen on dry ice. Twelve micron thick frontal sections are cut in a cryostat and mounted directly on SuperFrost™ Plus slides. Dried slides are fixed for 5 min in 4% paraformaldehyde. The slides are next rinsed 2×5 min in phosphate buffered saline (PBS; pH 7.4) followed by a brief acetylation: 500 μl acetic anhydride (100%) is added to 200 ml 0.1M triethanolamine and the slides immediately submerged for 2 min. Next, slides are passed through PBS twice (2×2 min) and finally through graded ethanol concentrations (30160/80/96/99/99) and allowed to dry. Immediately prior to hybridization the radioactively labeled probe is denatured for 3 min at 80° C. (denaturation) and mixed with hybridization buffer. The hybridization buffer consists of 50% deionised formamide, 1× SALTS (300 mM NaCl, 10 mM Tris, 10 mM NaPO₄ (pH 6.8), 5 mM EDTA, 0.02% Ficoll 400, 0.2% polyvinylpyrolidone (PVP-40, 40000 MW), 0.2% BSA Fraction V), 10% dextran sulphate, 1 μg/μl yeast tRNA and 9 mM DTT. Probe is added so that the final activity of the hybridization mix is approximately 15.000 cpm/μl. The hybridization mix is applied onto the sections (35 μl/section) that are cover-slipped. Hybridization is performed overnight at 47° C. The next day sections are subjected to two stringency washes at 62 and 67° C. Sections are washed for 1 hour at each temperature (lowest first) in a washing buffer consisting of 50% formamide, 1× SALTS and 10 mM DTT. The sections are next rinsed twice (2×2 min) in NTE buffer (0.5 M NaCl, 10 mM Tris-Cl (pH 7.2), 1 mM EDTA) containing 10 mM DTT, where after they are RNAse A treated (20 μg/ml; Boehringer-Mannheim) for 30 min. Next, the sections are rinsed twice for 5 min in NTE+10 mM DTT, 30 min in SSC (15 mM NaCl, 1.5 mM trisodiumcitrat, 2H₂O, pH=7.0) containing 1 mM DTT and finally dehydrated through a series of graded ethanol solutions containing 0.3M ammonium acetate (30/60/80/90/99). After drying the hybridized sections are exposed to Kodak bio-max film for 5 days prior to development. The film is developed using a standard four step procedure: 1) 5 min development in Kodak D19 developer, 2) 1 min in a stop bath (deionized water containing 1% acetic acid), 3) 5 min in fixative (G354, Ilford) and 4) 5-10 minutes rinse in deionized water.

Localization of Neuronatin mRNA in the Mouse and Rat Hypothalamus:

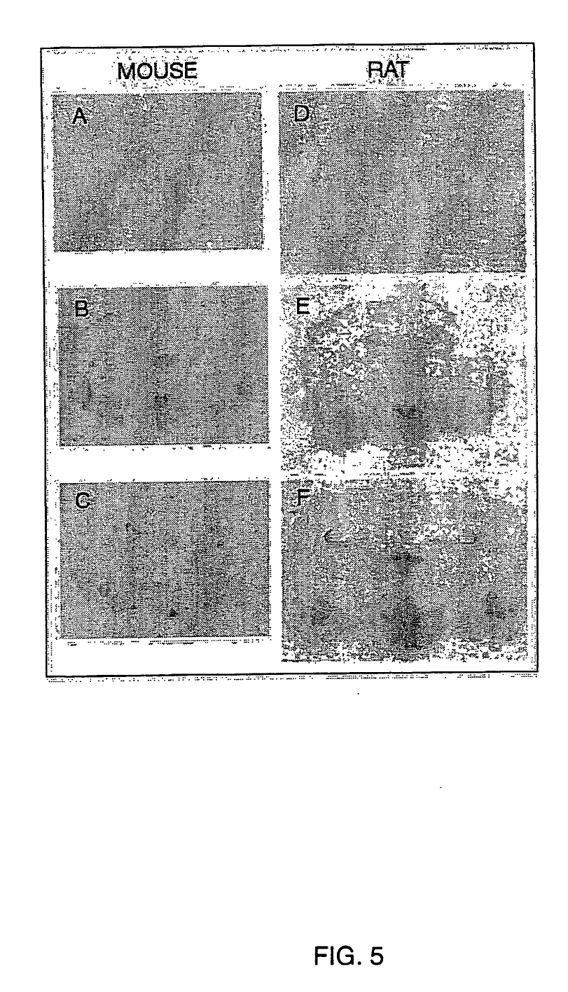

FIG. 5 shows autoradiograms of frontal hypothalamic sections from mouse (A, B, C) and rat (D, E, F). A and D are sections incubated with a sense cRNA probe and shows that no signal can be detected. Sections B and E are at the level of the paraventricular nucleus of the hypothalamus (PVN) and shows presence of neuronatin mRNA expression in this area. Further caudally in the hypothalamus the neuronatin gene is expressed in the arcuate, ventromedial, and dorsomedial (compact) hypothalamic nuclei as well as in the lateral hypothalamic areas. Outside the hypothalamus expression is seen in the amygdala, the habenula and in the hippocampus (see FIG. 5, C and F).

### Example 4

Neuronatin mRNA Expression Examined in Monogenetic Rodent Models of Obesity

Animals

Two separate experiments are performed using:

1) Eight obese Zucker fa/fa and 8 lean Fa/? littermates (Charles River, Sweden)

2) Twenty obese ob/ob and 9 lean Ob/? (C57BL/6J strain, Jackson Laboratories). All animals are housed under 12-h light, 12-h dark (lights on at 0600 h) in a temperature-controlled environment (22-24° C.) with free access to food and water. Rats are housed singly, mice 3-4 per cage. Two weeks after arrival in the laboratory the animals are assigned to their respective groups, and experiments start. Rats are decapitated in the morning (between 9-11 am), brains rapidly removed, frozen on dry ice and stored at −80° C. until further processing. The mice are divided in 3 groups: Group 1 (Ob/? veh) contains 9 animals and are injected with 0.5 ml PBS (pH 7.4, vehicle) daily for 5 days. Group 2 (ob/ob veh) contains 9 animals and are injected daily with vehicle (0.5 ml) for 5 days. Group 3 (ob/ob lep) contains 11 animals that are injected daily for 5 days with recombinant human leptin (1.5 mg/kg, Calbiochem-Novabiochem corporation, La Jolla, Calif.). On the morning of the sixth day the mice are killed by decapitation, brains rapidly removed and frozen on dry ice and kept at −80° C. until further processing. All brains are cut in a cryostat in the same fashion: 12 μm thick frontal sections through the hypothalamus are collected on SuperFrost™ slides (two sections per slide). Every tenth slide is counterstained with thionin and used to located specific areas. One slide from each animal containing the area of interest is processed for in situ hybridization with S35 labeled neuronatin antisense probes (in situ hybridization procedure described in Example 3). For each individual experiment (e.g. fa/fa vs. Fa/? rats, DMH-hybridization) all slides are processed simultaneously and exposed onto the same X-ray film. Autoradiograms are analyzed on a computerized image analysis system (Image 1.60b, NIH, USA), and the signal quantitated as the area of neuronatin expression (in the area of interest—DMH (Dorsomedial Hypothalamic Nucleus), PVN (Paraventricular Hypothalamic Nucleus), Arc (The Arcuate Nucleus), DMH+LHA (Lateral Hypothalamic Area) with a mean density above threshold multiplied with the mean of that area (Area=area×mean=arbitrary units). The average is taken of 2 sections per animal. For the rat experiments (containing two groups) an unpaired t-test is used to evaluate the results, for the mouse data one-way ANOVA with Newman-Keuls post-hoc test is applied. P values less than 0.05 are considered significant.

Neuronatin mRNA Levels are Downregulated in Animals with Defects in Leptin Signaling Pathways:

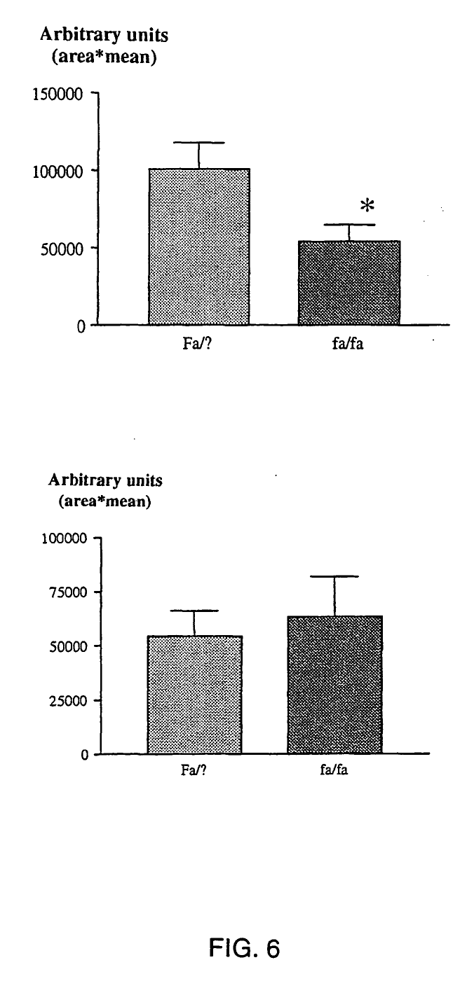

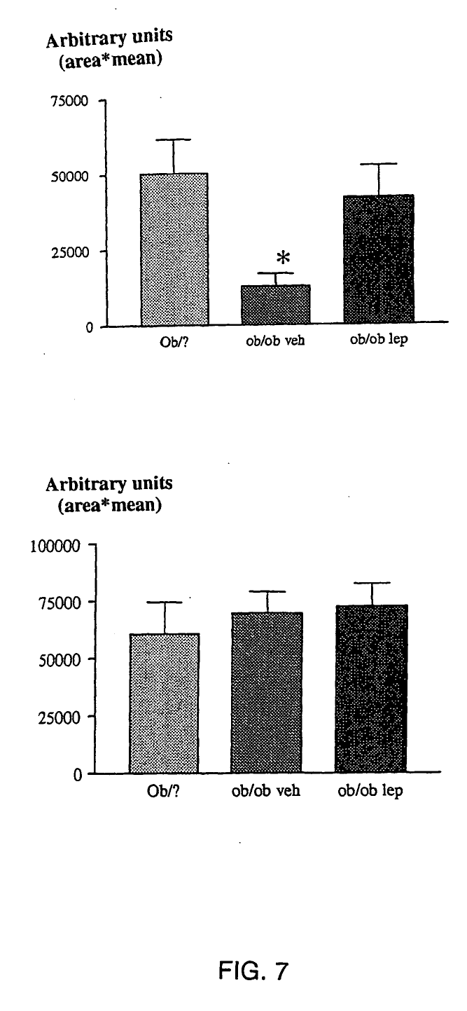

FIG. 6 shows the expression of neuronatin in fa/fa and Fa/? rats. In the DMH neuronatin expression is significantly lower in fa/fa compared to Fa/? rats (100900±17000 vs. 54210±10590, P<0.05). In contrast neuronatin expression does not differ in the Arc (fa/fa vs Fa/?; 54350±11830 vs. 63180±18660). Similarly, cf. FIG. 7, neuronatin is dowregulated in the DMH/LHA region in obese ob/ob mice compared to lean Ob/? mice (12806±4148 vs. 50351±11286, P<0.05). Five days of leptin treatment restores neuronatin expression in this region (ob/ob vehicle vs. ob/ob leptin; 12806±4148 vs 42000±10608, P<0.05).

### Example 5

Generation of Polyclonal Neuronatin Antibodies:

Peptides: Three different neuronatin fragments are used for the immunization procedure: Peptide 1 (Neuronatin alfa (aa 38-81)): the 44 amino-acid predicted neuronatin alpha-fragment (sequence: H-FRNPPGTQPIARSEVFRYSL-QKLAHTVSRTGRQVLGERRHRAPN-OH). Peptide 2: A 14 amino-acid neuronatin alpha-specific fragment (sequence: H-FRNPPGTOPIARSE-OH) and Peptide 3 (Neuronatin beta (aa 32-54)): The 23 amino-acid predicted neuronatin beta fragment (sequence: H-KLAHTVSRTGRQVLGERRHRAPN-OH). Peptides are coupled to bovine serum albumin (BSA fraction V; Roche Diagnostics) according to the following procedure: 1.8 mg peptide, 3.6 mg (BSA), 18 mg (1-ethyl-3-(3-dimethylaminopropyl))carbodiimide (Sigma), 0.6 ml N,N-dimethylformamide (Sigma) are mixed with 3.9 ml phosphate buffered saline (PBS, 50 mM) overnight. Twelve New Zealand White rabbits (Charles River, Sweden) housed under standard laboratory conditions with free access to food and water are used in the immunization experiments (4 rabbits injected with peptide 1, 4 with peptide 2 and 4 with peptide 3). Prior to immunization 20 ml of pre-immune blod is acquired from each rabbit. The first time rabbits are immunized with a mixture of 200 μl peptide with 300 μl Freunds complete adjuvant (Sigma). Booster injections consist of mixes of peptide and Freunds incomplete adjuvant (Sigma). Rabbits are injected every second week and bled every second week (alternate).

Immuno-Staining of Rat Brain Sections:

Twelve male SPD rats (300 g) housed under standard laboratory conditions are used for the experiments. The rats are anesthetized with 0.2 ml/100 g b.w of Hypnorm-Dormicum (1 ml contains: 0.167 mg fentanyl, 5 mg fluanisone, 2.5 mg Midazolam). The rats are next vascularly perfused with heparinized KPBS (15,000 IU/I), followed by 4% paraformaldehyde dissolved in 0.1 M phosphate buffer (pH=7.4) for 15 minutes. The brains are removed and postfixed in the same fixative over-night, cryoprotected for two days in a 30% sucrose-KPBS solution and cut in 40 μm thick frontal one-in-six series on a freezing microtome and collected in PBS. All reactions are carried out on free-floating sections. Serum from the immunized rabbits is used diluted 1:1000. Also pre-immune serum is used as controls diluted 1:1000. Immunohistochemistry is performed according to the following procedure: The sections are washed in KPBS for 3×10 minutes followed by 10 minutes incubation in 1% H₂O₂ in KPBS. Sections are then blocked for 20 minutes in 5% swine serum in KPBS containing 0.3% Triton X-100 (TX) and 1.0% bovine serum albumin (BSA). The serum is diluted in 0.3% TX and 1% BSA and sections incubate overnight at 4° C. The next day the sections are washed for 3×10 minutes in KPBS with 0.1% TX and (KPBS-T) before incubation for 60 minutes at room temperature in a biotinylated donkey anti-goat antibody (Jackson Immuno Research Lab., Inc.) diluted 1:2000 in KPBS-T. After another rinse for 3×10 minutes in KPBS-T followed by 60 minutes incubation in ABC-streptavidin horseradish peroxidase (Vector Elite Kit™) the sections were washed for 3×10 minutes in KPBS-T before being developed in Chromagen Solution for 2-20 minutes (0.04% DAB+0.003% H₂O₂ in KPBS). Immunostaining is evaluated on a Nikon microscope and images acquired by a Nikon DCM1200 digital camera.

Results:

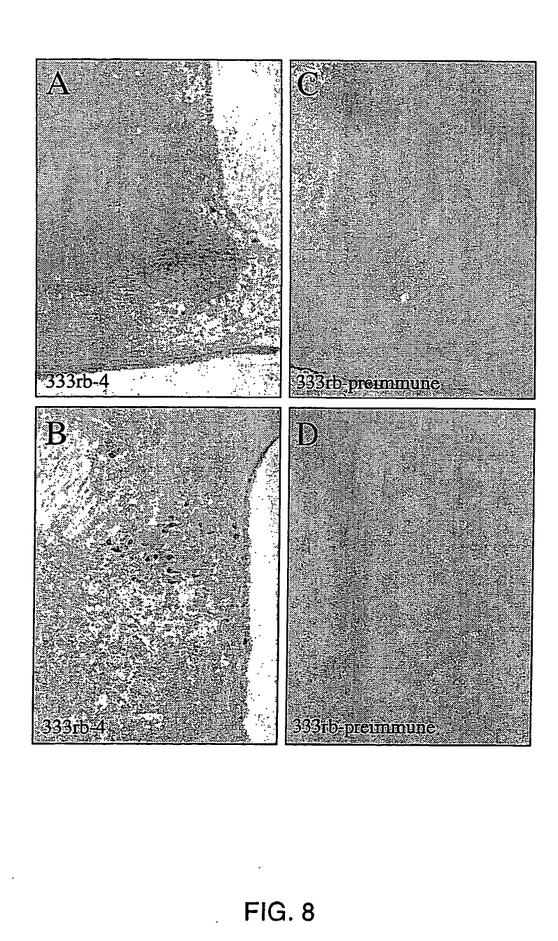

Three rabbits yielded serum that stained cells in the hypothalamus. Rabbit 333rb immunized with Peptide 1 (neuronatin alpha fragment) is further characterized. FIG. 8 shows the staining obtained using serum from rabbit 333rb (4^{th} bleed). Staining is observed in hypothalamic areas exactly matching the signal observed in the in situ hybridization experiments. FIGS. 8A and B shows staining of cells in the suprachiasmatic nucleus (SCN) and in the paraventricular hypothalamic nucleus (PVN). This staining pattern is not observed using pre-immune serum from the same rabbit (333rb-preimmune, FIG. 8, C and D (sections matching A and B)).

### Example 6

Characterization of Neuronatin Peptides by Western Blotting

Extraction of Protein

Rat hypothalamic tissue frozen on dry ice is extracted with 500 μl solubilization buffer (1.25×LDS sample buffer (Novex Invitrogen, NP0007), 62.5 mM dithiotreitol, 25 pvml Protease inhibitor cocktail for mammalian cells (Sigma, P8340) per 125 mg tissue by solubilization with a rotor-stator-type blender. Samples are denatured by heating to 70 degrees for 10 min, cooled to room temperature, and treated with 2.5 μl benzonase (VWR, 1654) for 15 min. The lysate is cleared by centrifuging 10 minutes at 10000 g and the proteins in the supernatant precipitated by the addition of 8 volumes −20° C. acetone and storage at −20° C. overnight. The precipitate is collected by centrifuging 15 minutes at 3000 g, 4° C., is dried briefly, and is solubilized in 1×LDS sample buffer with 50 mM dithiotreitol.

The protein concentration is determined by using a Bradford kit (Bio-Rad, 500-0001) as described by the manufacturer.

Western Blotting and ECL

Samples containing 50 μg hypothalamic protein with 10 ng neuronatin α, 10 ng neuronatin beta, 50 μg hypothalamic protein, and 10 ng neuronatin alpha, respectively, are run on a 12% Nupage gel (Invitrogen Novex, NP0342) using the MES buffer system (Invitrogen Novex, NP0002) followed by blotting onto PVDF membrane (Invitrogen Novex, LC2002) as described by the manufacturer.

Neuronatin peptides are detected by enhanced chemoluminiscense using the following procedure: The membrane is blocked overnight in 1×PBS, 10% nonfat dry milk, and 0.1% Tween 20) at 4° C. The membrane is quickly washed in PBS and incubated with Neuronatin antibody (333rb-4) diluted 1:250 in PBS with 5% nonfat dry milk for 60 min. at room temp. The membrane is quickly washed in PBS followed by 6 washes (5 min. each) in PBS with 0.1% Tween. The membrane is incubated in horseradish peroxidase coupled antirabbit IgG (Cell Signaling, 7074) diluted 1:60000 in PBS with 5% nonfat dry milk for 60 min. at room temp and washed as above. Detection is performed with the Supersignal West Femto maximum sensitivity substrate (Pierce, 34095) as described by the manufacturer.

Results:

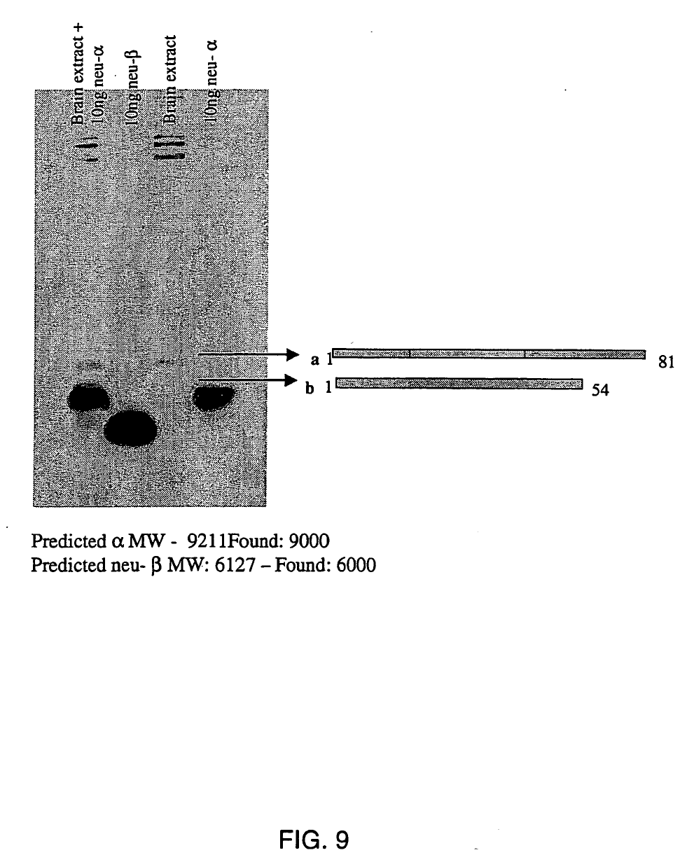

The antibody used for the immunohistochemistry experiments (333rb-4) is used for detection of neuronatin polypeptides in Western blots of protein extracts from the hypothalamus, cf. FIG. 9. The antibody recognizes the alfa-form (against which it was generated) and also the beta form (lanes 1, 2 and 4). Pure extract gives rise to two bands that, based on size, appear to be the unprocessed alpha and beta forms. The processed alpha and beta forms of Neuronatin are not detectable in the hypothalamic extract.

### Example 7

Acute Effects of Peptide 1 (Neuronatin α) on Food Intake and Activity

Animals and Surgery:

In this experiment Thirty-two male Sprague Dawley rats (Charles River, Germany) weighing between 250 and 275 grams are used. The animals are kept on a 12/12 L/D cycle lights on 3:00AM in temperature-controlled environment in single cages with ad libitum access to powdered chow and water. All experiments are conducted in accordance with internationally accepted principles for the care and use of laboratory animals and are approved by the Danish committee for animal research. Under Hypnorm Dormicum (Nomeco A/S, Copenhagen Denmark) anesthesia all animals are stereotaxically equipped with a stainless steel cannula aimed at the lateral ventricle (1 mm caudal and 1.5 mm lateral from bregma and 4 mm depth). During a 7 day recovery period, the rats are handled daily in order to accustom them to the experimental procedure. Two days postoperatively the animals are placed in customized metabolic cages with online registration of food intake and activity (MaNi FeedWin, Ellegaard systems A/S Faaborg Denmark). At day seven postoperatively the animals are subjected to a standard cross-over design with three doses: a) Peptide 1 (as defined in Example 5) 20 μg/10 μl icv, b) Peptide 1 40 μg/10 μl icv and c) vehicle 10 μl. The animals are injected intracerebroventricularly (icv) just prior to lights out (1400 h). Food intake and activity is measured every 15 minutes for 15 hours. The animals are allowed a two-day drug free period until the next experiment.

Results:

Food Intake

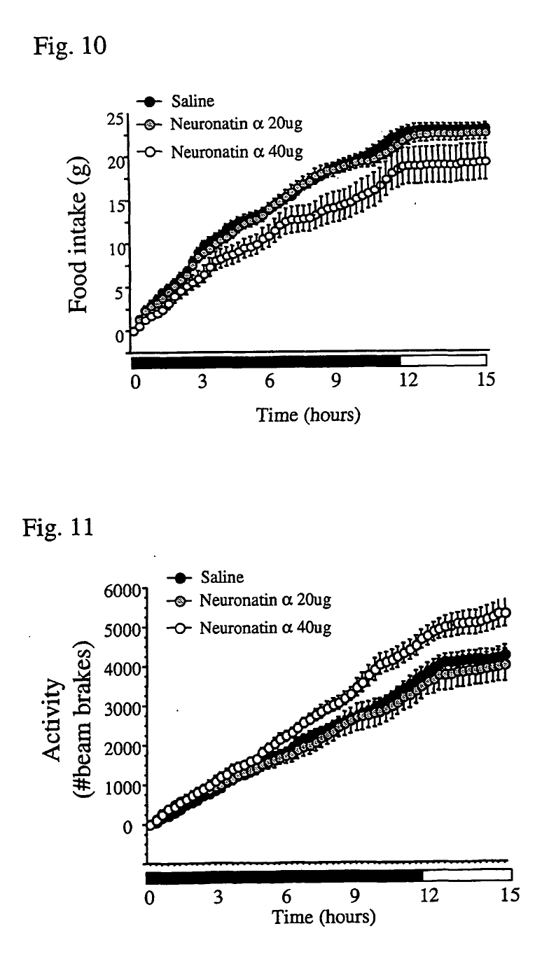

Central administration of 40 μg Peptide 1 into the lateral ventricle results in a reduction in food intake present 3 hours after the injection (FIG. 10). This reduction is statistically significant using a one-way ANOVA followed by Fischer's post hoc test when compared to vehicle as well as 20 μg Peptide 1. The reduction in food intake lasts until 1 hour before lights on (11 hours after the injection). The 40 μg dose of Peptide 1 has no long-term effects on food intake as the rats catch up the following day (data not shown). Reducing the dose to 50% (20 μg) does not result in any reduction of food intake.

Activtiy and Behavioural Specificity

Central administration of 40 μg Peptide 1 into the lateral ventricle results in an increase in locomotor activity (assessed as consecutive beam breaks of infrared photo beams scattered over the test cage) present approximately 5 hours after the injection (FIG. 11). This reduction is statistically significant using a one-way ANOVA followed by Fischer's post hoc test when compared to vehicle as well as 20 μg Peptide 1. The increase in activity is seen throughout the remainder of the test period—15 hours after the injection. There is no prolonged effect of 40 μg of Peptide 1 on activity (data not shown). 20 μg Peptide 1 has no effect on locomotor behavior. The decrease in food intake after 40 μg Peptide 1 is not due to general malaise since this dose does not produce a conditioned taste aversion (data not shown).

### Example 8

The Acute Effect of Peptide 3 (Neuronatin Beta) on Food Intake and Activity

Animals and Surgery:

In this experiment 32 male Sprague Dawley rats (Charles River, Germany) weighing between 250 and 275 grams are used. Operations and housing as Example 7. Two days postoperatively the animals are placed in customized metabolic cages with online registration of food intake and activity (MaNi FeedWin, Ellegaard systems A/S Faaborg Denmark). At day seven postoperatively the animals are subjected to a standard cross-over design with two doses: a) Peptide 3 (as defined in Example 5) 40 μg/10 μl icv or b) vehicle 10 μl. The animals are injected icv just prior to lights out (1400 h). Food intake and activity is measured in 15 minutes bins for a 15-hour period.

Results:

Food Intake

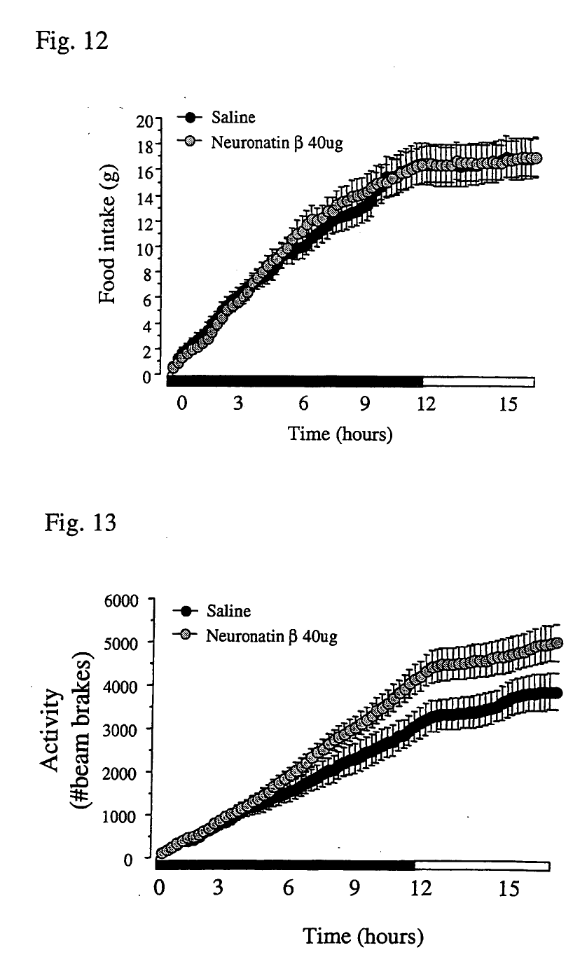

Central administration of 40 μg Peptide 3 into the lateral ventricle results does not affect food intake during the first 15 hours (FIG. 12) when compared to vehicle.

Activtiy

Central administration of 40 μg Peptide 3 into the lateral ventricle results in an increase in locomotor activity that presents approximately 5 hours after the injection (FIG. 13). This reduction is statistically significant using a one-way anova followed by Fischer's post hoc test when compared to vehicle. The increase in activity is seen throughout the remainder of the test period—15 hours after the injection. There are no prolonged effects of 40 μg of Peptide 3 on activity (data not shown).

### Example 9

The chronic effect of Peptide 1 (Neuronatin alfa) on food intake and body weight. In this experiment 32 male Sprague Dawley rats (Charles River, Germany) weighing between 250 and 275 grams are used. Operations and housing as in Example 7. Two days postoperatively the animals are placed in customized metabolic cages with online registration of food intake and activity (MaNi FeedWin, Ellegaard systems A/S Faaborg Denmark). On day seven postoperatively the animals are randomized into three groups: a) Peptide 1 (as defined in Example 5) 20 μg/10 μl icv, b) Peptide 1 40 μg/10 μl icv and c) vehicle 10 μl. The animals are injected twice a day at 6:30 am and just prior to lights out (3:30 pm). Food intake and activity is measured in one hour bins for a 7 days period. Fluid intake and body weight is measured on a daily basis. After the 7 days on this regimen animals are killed by an overdose of CO₂ followed by cervical dislocation.

Results:

Body Weight

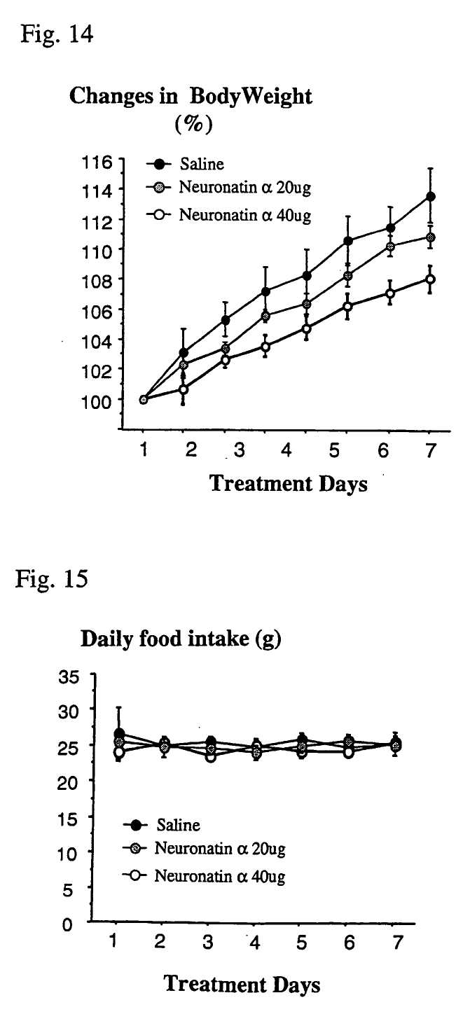

Central administration of Peptide 1 twice daily significantly reduces body weight gain (day of study start is set to 100; FIG. 14. At day 7 Peptide 1 treated rats receiving 40 μg twice daily have an increased body weight by 7.2±0.8% (significantly different from the saline treated group (11.5±1.4%) and the 20 μg Peptide 1 treated group (10.3±0.7%)); P<0.05).

Food Intake

Neither 40 μg nor 20 μg Peptide 1 has any effect on food intake over the seven days treatment period (FIG. 15).

1. A method of preventing or treating a metabolic disorder of an animal comprising administering to the animal an effective amount of 1) a polypeptide according to SEQ ID NO: 3 (α-neuronatin) or SEQ ID NO: 4 (β-neuronatin), 2) a biologically active variant of one of the said sequences having at least 50% identity therewith, 3) a biologically active fragment of SEQ ID NO: 3 or SEQ ID NO: 4 comprising at least 7 amino acids, or 4) a biologically active variant of the said fragment having at least 80% identity therewith.

2. A method according to any of the preceding claims, wherein the said fragment of SEQ ID NO: 3 is the fragment fragment consisting of amino acids 38-81, and the said fragment of SEQ ID NO: 4 is the fragment consisting of amino acids 32-54.

3. A method according to claim 1 or 2, wherein the said fragment comprises at least 9, preferably 15, more preferably 27, more preferably 39 and most preferably 51 amino acids.

4. A method according to claim 1, wherein the variant of SEQ ID NO: 3 or SEQ ID NO: 4 has at least 60%, preferably 70%, more preferably 80% and most preferably 90% identity therewith.

5. A method according to claim 1, wherein the variant of the said fragment has at least 85%, preferably 90% and most preferably 95% identity therewith.

6. A method of preventing or treating a metabolic disorder in an animal comprising administering to the animal an effective amount of a biologically active polypeptide encoded by 1) a polynucleotide according to SEQ ID NO: 1 α-neuronatin) or SEQ ID NO: 2 (β-neuronatin), 2) a variant of one of the said sequences having at least 30% identity therewith, 3) a fragment of SEQ ID NO: 1 or SEQ ID NO: 2 comprising at least 21 nucleotides, 4) a variant of one of the said fragments having at least 60% identity therewith, or 5) a sequence, which hybridises with SEQ ID NO: 1 or SEQ ID NO: 2 under highly stringent conditions.

7. A method according to claim 1, wherein the metabolic disorder is the disease complex called metabolic syndrome X.

8. A method according to claim 1, wherein the metabolic disorder is a body weight disorder or non-insulin dependent diabetes.

9. A method according to claim 1 comprising administering to the animal an effective amount of an anti-sense polynucleotide to SEQ ID NO: 1 or SEQ ID NO: 2.

10. A polypeptide having the sequence of 1) the fragment of SEQ ID NO: 3 consisting of amino acids 38-81, 2) the fragment of SEQ ID NO: 4 consisting of amino acids 32-54, or 3) a biologically active variant of one of the said sequences having at least 50% identity therewith.

11. A polypeptide for preventing or treating a metabolic disorder in an animal, wherein the polypeptide is 1) a polypeptide according to SEQ ID NO: 3 (α-neuronatin) or SEQ ID NO: 4 (β-neuronatin), 2) biologically active a variant of one of the said sequences having at least 50% identity therewith, 3) a biologically active fragment of SEQ ID NO: 3 or SEQ ID NO: 4 comprising at least 7 amino acids, or 4) a biologically active variant of the said fragment having at least 80% identity therewith.

12. Use of 1) a polypeptide according to SEQ ID NO: 3 (α-neuronatin) or SEQ ID NO: 4 (β-neuronatin), 2) a biologically active variant of one of the said sequences having at least 50% identity therewith, 3) a biologically active fragment of SEQ ID NO: 3 or SEQ ID NO: 4 comprising at least 7 amino acids, or 4) a biologically active variant of the said fragment having at least 80% identity therewith, for the manufacture of a pharmaceutical composition for preventing or treating a metabolic disorder in an animal.

13. A pharmaceutical composition comprising as an active substance 1) a polypeptide according to SEQ ID NO: 3 α-neuronatin) or SEQ ID NO: 4 (β-neuronatin), 2) a biologically active variant of one of the said sequences having at least 50% identity therewith, 3) a biologically active fragment of SEQ ID NO: 3 or SEQ ID NO: 4 comprising at least 7 amino acids, or 4) a biologically active variant of the said fragment having at least 80% identity therewith.

14. A pharmaceutical composition for preventing or treating metabolic disorders comprising as an active substance 1) a polypeptide according to SEQ ID NO: 3 (α-neuronatin) or SEQ ID NO: 4 (β-neuronatin), 2) a biologically active variant of one of the said sequences having at least 50% identity therewith, 3) a biologically active fragment of SEQ ID NO: 3 or SEQ ID NO: 4 comprising at least 7 amino acids, or 4) a biologically active variant of the said fragment having at least 80% identity therewith.

15. A pharmaceutical composition comprising as an active substance a stimulator to a polypeptide according to SEQ ID NO: 3 (α-neuronatin) or SEQ ID NO: 4 (β-neuronatin), or a natural occurring variant thereof.

16. A pharmaceutical composition according to claim 15, wherein the stimulator is a small molecule.

17. A pharmaceutical composition comprising as an active substance an inhibitor to a polypeptide according to SEQ ID NO: 3 (α-neuronatin) or SEQ ID NO: 4 (β-neuronatin), or a natural occurring variant thereof.

18. A pharmaceutical composition according to claim 16, wherein the inhibitor is an antibody to the said polypeptide.

19. A pharmaceutical composition according to claim 16, wherein the inhibitor is a small molecule.

20. A pharmaceutical composition comprising as an active substance an inhibitor to a polynucleotide according to SEQ ID NO: 1 (α-neuronatin) or SEQ ID NO: 2 (β-neuronatin), or a natural occurring variant thereof.

21. A pharmaceutical composition according to claim 20, wherein the inhibitor is an anti-sense polynucleotide to SEQ ID NO: 1 or SEQ ID NO: 2.

22. A method of diagnosing or prognosticating a metabolic disorder in an animal comprising determining the sequence of the polynucleotide, which encodes neuronatin, by the use of 1) a polynucleotide according to SEQ ID NO: 1 α-neuronatin) or SEQ ID NO: 2 (β-neuronatin), 2) a variant of one of the said sequences having at least 30% identity therewith, 3) a fragment of SEQ ID NO: 1 or SEQ ID NO: 2 comprising at least 21 nucleotides, 4) a variant of one of the said fragments having at least 60% identity therewith, or 5) a sequence, which hybridises with SEQ ID NO: 1 or SEQ ID NO: 2 under highly stringent conditions, and comparing the sequence determined with SEQ ID NO: 1 and/or SEQ ID NO: 2 to identify differences in the sequence.

23. A method of diagnosing or prognosticating a metabolic disorder in an animal comprising determining the level of neuronatin in a biological sample using an antibody to neuronatin, and using the measurement to evaluate the state of the animal.

24. An antibody to 1) a polypeptide according to SEQ ID NO: 3 α-neuronatin) or SEQ ID NO: 4 (β-neuronatin), 2) a biologically active variant of one of the said sequences having at least 50% identity therewith, 3) a biologically active fragment of SEQ ID NO: 3 or SEQ ID NO: 4 comprising at least 7 amino acids, or 4) a biologically active variant of the said fragment having at least 80% identity therewith.

25. A method of identifying an interaction partner to neuronatin comprising using 1) a polypeptide according to SEQ ID NO: 3 (α-neuronatin) or SEQ ID NO: 4 (β-neuronatin), 2) a biologically active variant of one of the said sequences having at least 50% identity therewith, 3) a biologically active fragment of SEQ ID NO: 3 or SEQ ID NO: 4 comprising at least 7 amino acids, or 4) a biologically active variant of the said fragment having at least 80% identity therewith, to screen an expression library for interaction partners.

26. A method of identifying an interaction partner to neuronatin comprising using 1) a polynucleotide according to SEQ ID NO: 1 (α-neuronatin) or SEQ ID NO: 2 (β-neuronatin), 2) a variant of one of the said sequences having at least 30% identity therewith, 3) a fragment of SEQ ID NO: 1 or SEQ ID NO: 2 comprising at least 21 nucleotides, 4) a variant of one of the said fragments having at least 60% identity therewith, or 5) a sequence, which hybridises with SEQ ID NO: 1 or SEQ ID NO: 2 under highly stringent conditions, to screen an expression library for interaction partners.

27. A vector construct comprising 1) a polynucleotide according to SEQ ID NO: 1 (α-neuronatin) or SEQ ID NO: 2 (β-neuronatin), 2) a variant of one of the said sequences having at least 30% identity therewith, 3) a fragment of SEQ ID NO: 1 or SEQ ID NO: 2 comprising at least 21 nucleotides, 4) a variant of one of the said fragments having at least 60% identity therewith, or 5) a sequence, which hybridises with SEQ ID NO: 1 or SEQ ID NO: 2 under highly stringent conditions, and a promoter operably linked to the polynucleotide.

28. The vector construct according to claim 27 being a viral vector, an adenoviral vector, an adenovirus-associated viral vector, a lentivirus vector, a retroviral vector or a vacciniaviral vector.

29. A packaging cell line capable of producing an infective virion comprising the vector of claim 27 or 28.

30. A recombinant host cell comprising 1) a polynucleotide according to SEQ ID NO: 1 (α-neuronatin) or SEQ ID NO: 2 (β-neuronatin), 2) a variant of one of the said sequences having at least 30% identity therewith, 3) a fragment of SEQ ID NO: 1 or SEQ ID NO: 2 comprising at least 21 nucleotides, 4) a variant of one of the said fragments having at least 60% identity therewith, or 5) a sequence, which hybridises with SEQ ID NO: 1 or SEQ ID NO: 2 under highly stringent conditions, or the vector of either of claim 27 or 28.

31. The host cell of claim 30, which is a human cell, a dog cell, a monkey cell, a rat cell or a mouse cell.

32. A pharmaceutical composition comprising 1) a polynucleotide according to SEQ ID NO: 1 (α-neuronatin) or SEQ ID NO: 2 (β-neuronatin), 2) a variant of one of the said sequences having at least 30% identity therewith, 3) a fragment of SEQ ID NO: 1 or SEQ ID NO: 2 comprising at least 21 nucleotides, 4) a variant of one of the said fragments having at least 60% identity therewith, or 5) a sequence, which hybridises with SEQ ID NO: 1 or SEQ ID NO: 2 under highly stringent conditions, the vector of claim 27 or 28, the packaging cell line of claim 29 or the host cell of claim 30 or 31.

33. A method of preventing or treating a metabolic disorder in an animal comprising administering to the animal a 1) a polynucleotide according to SEQ ID NO: 1 α-neuronatin) or SEQ ID NO: 2 (β-neuronatin), 2) a variant of one of the said sequences having at least 30% identity therewith, 3) a fragment of SEQ ID NO: 1 or SEQ ID NO: 2 comprising at least 21 nucleotides, 4) a variant of one of the said fragments having at least 60% identity therewith, or 5) a sequence, which hybridises with SEQ ID NO: 1 or SEQ ID NO: 2 under highly stringent conditions, the vector of claim 27 or 28, the packaging cell line of claim 29 or the host cell of claim 30 or 31.

34. A method of effecting an increase in locomotor activity and/or a reduction in food intake in an animal comprising administering to the animal an effective amount of 1) a polypeptide according to SEQ ID NO: 3 (α-neuronatin) or SEQ ID NO: 4 (β-neuronatin), 2) a biologically active variant of one of the said sequences having at least 50% identity therewith, 3) a biologically active fragment of SEQ ID NO: 3 or SEQ ID NO: 4 comprising at least 7 amino acids, or 4) a biologically active variant of the said fragment having at least 80% identity therewith.


SEQUENCE LISTING (9 sequences)

SEQ ID NO: 1 - DNA, 1281 bp, Homo sapiens
  misc_feature: Identity a-neuronatin - Coding sequence 71-316 - Genbank no. BC001768
agcggactccgagaccagcggatctcggcaaaccctctttctcgaccacccacctaccattcttggaaccatggcggcagtggcggcggcctcggctgaactgctcatcatcggctggtacatcttccgcgtgctgctgcaggtgttcctggaatgctgcatttactgggtaggattcgcttttcgaaatcctccagggacacagcccattgcgagaagtgaggtgttcaggtactccctgcagaagctggcatacacggtgtcgcggaccgggcggcaggtgttgggggagcgcaggcagcgagcccccaactgaggccccagctcccagccctgggcggccgtatcatcaggtgctcctgtgcatctcggccagcacgggagccagtgccgcgcaggaatgtggggtcccctgtgttccctcgccagaggagcacttggcaaggtcagtgaggggccagtagacccccggagaagcagtaccgacaatgacgaagataccagatcccttcccaacccctttgcaccggtcccactaaggggcagggtcgagagaggaggggggatagggggagcagacccctgagatctgggcataggcaccgcattctgatctggacaaagtcgggacagcaccatcccagccccgaagccagggccatgccagcaggccccaccatggaaatcaaaacaccgcaccagccagcagaatggacattctgacatcgccagccgacgccctgaatcttggtgcagcaccaaccgcgtgcctgtgtggcgggactggagggcacagttgaggaaggagggtggttaagaaatacagtggggccctctcgctgtcccttgcccagggcacttgcattccagcctcgctgcatttgctctctcgattcccctttcctcctcactgcctcccaagcccaccctactccaaaataatgtgtcacttgatttggaactattcaagcagtaaaagtaaatgaatcccacctttactaaaacactttctctgaaccccccttgcccctcactgatcttgcttttccctggtctcatgcagttgtggtcaatattgtggtaatcgctaattgtactgattgtttaagtgtgcattagttgtgtctccccagctagattgtaagctcctggaggacagggaccacctctacaaaaaataaaaaaagtacctcccctgtctcgcacagtgtcccaggaccctgcggtgcagtagaggcgcaccaaaaaaaaaaaaaaaaaaaaaaaaaaaaaaaaaaaaaa

SEQ ID NO: 2 - DNA, 1158 bp, Homo sapiens
  misc_feature: Identity b-neuronatin - Coding sequence 72-236 - Genbank no. U25034
cagcggactccgagaccagcggatctcggcaaaccctctttctcgaccacccacctaccattcttggaaccatggcggcagtggcggcggcctcggctgaactgctcatcatcggctggtacatcttccgcgtgctgctgcaggtgttcaggtactccctgcagaagctggcatacacggtgtcgcggaccgggcggcaggtgttgggggagcgcaggcagcgagcccccaactgaggccccagctcccagcctgggcggccgtatatagtgctcctgtgcatctcggccagcacgggagccagtgccgcgcaggaatgtggggtcccctgtgttccctcgccagaggagcacttggcaaggtcagtgaggggccagtagacccccggagaagcagtaccgacaatgacgaagataccagatcccttcccaacccctttgcaccggtcccactaaggggcagggtcgagagaggaggggggatagggggagcagaccctgagatctgggcataggcaccgcattctgatctggacaaagtcgggacagcaccatcccagccccgaagccagggccatgccagcaggccccaccatggaaatcaaaacaccgcaccagccagcagaatggacattctgacatcgccagccgacgccctgaatcttggtgcagcacccaccgcgtgcctgtgtggcgggactggagggcacagttgaggaaggagggtggttaagaaatacagtggggccctctcgctgtcccttgcccagggcacttgtattccagcctcgctgcatttgctctctcgattgcccctttcctcctacatgcctcccaagcccaccctactccaaaagtaatgtgtcacttgatttggaactattcaagcagtaaaagtaaatgaatcccacctttactaaaacactttctctgaaccccccttgcccctcactgatcttgcttttccctggtctcatgcagttgtggtcaatattgtggtaatcgctaattgtactgattgtttaagtgtgcattagttgtctctccccagctagattgtaagctcctggaggacagggaccacctctacaaaaaataaaaaaagtacctcccctgtctcgcacagtgtcccaggaccctgcggtgcagtagaggcgca

SEQ ID NO: 3 - protein, 81 aa, Homo sapiens
  MISC_FEATURE: Identity a-neuronatin - Genbank no. BC001768
MAAVAAASAELLIIGWYIFRVLLQVFLECCIYWVGFAFRNPPGTQPIARSEVFRYSLQKLAYTVSRTGRQVLGERRQRAPN

SEQ ID NO: 4 - protein, 54 aa, Homo sapiens
  MISC_FEATURE: Identity b -neuronatin - Genbank no. U25034
MAAVAAASAELLIIGWYIFRVLLQVFRYSLQKLAYTVSRTGRQVLGERRQRAPN

SEQ ID NO: 5 - DNA, 84 bp, Artificial sequence
  Synthetic oligonucleotide which shows 100% identity to the 3′ end of the neuronatin gene
gagatatgggcgtaggcaccacattctgatctggaccaagtcggaacagtaccatctcagccgcacaagatcctaccatgaaga

SEQ ID NO: 6 - DNA, 20 bp, Artificial sequence
  5′ primer directed against the murine neuronatin cDNA sequence
catcaaccagcagaatggac

SEQ ID NO: 7 - DNA, 20 bp, Artificial sequence
  3′ primer directed against the murine neuronatin cDNA sequence
tgagaccagggataagcaag

SEQ ID NO: 8 - protein, 44 aa, Artificial sequence
  Neuronatin alpha fragment
FRNPPGTQPIARSEVFRYSLQKLAHTVSRTGRQVLGERRHRAPN

SEQ ID NO: 9 - protein, 23 aa, Artificial sequence
  Neuronatin beta fragment
KLAHTVSRTGRQVLGERRHRAPN